SEQUENCE LISTING (8 sequences)

SEQ ID NO: 1 - DNA, 13143 bp, Artificial sequence
  Exemplary plasmid P273 comprising TCR beta variable, diversity and joining gene sequences with spacers for V(D)J recombination
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaccggggagtcccttttaggcacttgcttctggtgctgcaactggcgctcctcccagcagccactcagggaaagaaagtggtgctgggcgggaaacccattcccaatcccctccttgggcttgactccacccggacgggaggtggcggaggctccggcaagcctatccctaaccctctcctcggcctcgattctacgcgtaccggtggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggtgtgacgatctgatcaagagacaggataaggagccgccaccatggagtttgggctgagctggctttttcttgtggctattttaaaaggtgtccagtgtgagcctgaagtgacccagacccctagccaccaagtgacacagatgggccaggaagtgatcctgcgctgcgtgcccatcagcaaccacctgtacttctactggtacagacagatcctggggcagaaagtggaatttctggtgtccttctacaacaacgagatcagcgagaagtccgagatcttcgacgaccagttcagcgtggaacggcccgacggcagcaacttcaccctgaagatcagaagcaccaagctggaagatagcgccatgtacttctgtgccagcagtgaagccacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagccaagtgcaaagggacaggaggtttttgttaagggctgtatcactgtggggacagggggccacagtgatacagcccttaacaaaaacccctactgcaacctggcggtaaacccctatttgtttatttttctaaatacattcaaatatgtatccgctcatgagacaataaccctgataaatgcttcaataatattgaaaaaggaagagtatggcgaaactgaccagcgcggtgccggttctgaccgcgcgtgatgtggcgggtgcggtggaattttggaccgatcgtctgggctttagccgtgattttgtggaagatgattttgcgggcgtggtgcgtgatgatgtgaccctgtttattagcgcggtgcaggatcaggtggtgccggataacaccctggcctgggtgtgggtgcgtggcctggatgaactgtatgcggaatggtctgaagtggtgagcaccaactttcgtgatgcgagcggtccggccatgaccgaaattggcgaacagccgtggggccgtgaatttgcgctgcgtgatccggcgggtaactgcgtgcattttgtggcggaagaacaggattaataactgtggttggaaccttagatccggaggccagcccttctcatgttcagagaacatggttaactggttaagtcatgtcgtcccacaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgtgaacactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccctagatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggaaagccccagcggagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccagagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttgaccgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagagacaagctggctagcgtacatactgaagcttgccgccaccatgacttccaagctggccgtggctctcttggcagccttcctgatttctgcagctctgtgtaagaacgaggtggaacagagcccccagaacctgaccgcccaggaaggcgagttcatcaccatcaactgcagctacagcgtgggcatcagcgccctgcattggctgcagcagcatcctggcggcggaatcgtgtccctgttcatgctgagcagcggcaagaagaagcacggccggctgatcgccaccatcaatatccaggaaaagcacagcagcctgcacatcaccgccagccaccctagagacagcgccgtgtacatttgcgccgtgcggaccaacaccggcaaccagttctacttcggcaccggcaccagcctgaccgtgatccctaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaatcctttgaaacagatacgaacctaaactttcaaaacctgtcactagatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggaaagccccagcggagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccagataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggactcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 12561 bp, Artificial sequence
  Exemplary plasmid P262 comprising TCR beta variable, diversity and joining gene sequences with spacers for V(D)J recombination
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaccggggagtcccttttaggcacttgcttctggtgctgcaactggcgctcctcccagcagccactcagggaaagaaagtggtgctgggcgggaaacccattcccaatcccctccttgggcttgactccacccggacgggaggtggcggaggctccggcaagcctatccctaaccctctcctcggcctcgattctacgcgtaccggtggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggtgtgacgatctgatcaagagacaggataaggagccgccaccatggagtttgggctgagctggctttttcttgtggctattttaaaaggtgtccagtgtgagcctgaagtgacccagacccctagccaccaagtgacacagatgggccaggaagtgatcctgcgctgcgtgcccatcagcaaccacctgtacttctactggtacagacagatcctggggcagaaagtggaatttctggtgtccttctacaacaacgagatcagcgagaagtccgagatcttcgacgaccagttcagcgtggaacggcccgacggcagcaacttcaccctgaagatcagaagcaccaagctggaagatagcgccatgtacttctgtgccagcagtgaagccacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagccaagtgcaaagggacaggaggtttttgttaagggctgtatcactgtggggacagggggccacagtgatacagcccttaacaaaaacccctactgcaacctggcggtaaacccctatttgtttatttttctaaatacattcaaatatgtatccgctcatgagacaataaccctgataaatgcttcaataatattgaaaaaggaagagtatggcgaaactgaccagcgcggtgccggttctgaccgcgcgtgatgtggcgggtgcggtggaattttggaccgatcgtctgggctttagccgtgattttgtggaagatgattttgcgggcgtggtgcgtgatgatgtgaccctgtttattagcgcggtgcaggatcaggtggtgccggataacaccctggcctgggtgtgggtgcgtggcctggatgaactgtatgcggaatggtctgaagtggtgagcaccaactttcgtgatgcgagcggtccggccatgaccgaaattggcgaacagccgtggggccgtgaatttgcgctgcgtgatccggcgggtaactgcgtgcattttgtggcggaagaacaggattaataactgtggttggaaccttagatccggaggccagcccttctcatgttcagagaacatggttaactggttaagtcatgtcgtcccacaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgtgaacactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccctagatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggaaagccccagcggagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccagagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttctggctccggcagacgcagaagaagaagatctggcagcggcgccaccaacttcagcctgctgaaacaggccggggatgtggaagagaaccctggccctagcggcatgacttccaagctggccgtggctctcttggcagccttcctgatttctgcagctctgtgtaagaacgaggtggaacagagcccccagaacctgaccgcccaggaaggcgagttcatcaccatcaactgcagctacagcgtgggcatcagcgccctgcattggctgcagcagcatcctggcggcggaatcgtgtccctgttcatgctgagcagcggcaagaagaagcacggccggctgatcgccaccatcaatatccaggaaaagcacagcagcctgcacatcaccgccagccaccctagagacagcgccgtgtacatttgcgccgtgcggaccaacaccggcaaccagttctacttcggcaccggcaccagcctgaccgtgatccctaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaatcctttgaaacagatacgaacctaaactttcaaaacctgtcactagatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggaaagccccagcggagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgccccccagataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaattcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggaaagcttgccgccaccatggacccccccagagccagccacctgagcccccggaagaagcggcccagacagacaggcgccctgatggccagcagcccccaggacatcaagttccaggacctggtggtgttcatcctggaaaagaagatgggcaccaccagacgggcctttctgatggaactggccagacggaagggcttccgggtggagaacgagctgtccgacagcgtgacccacatcgtggccgagaacaacagcggcagcgacgtgctcgaatggctgcaggcccagaaagtgcaggtgtccagccagcccgagctgctggacgtgtcctggctgatcgagtgcatcagagccggcaagcccgtggagatgaccggcaagcaccagctggtcgtgcggcgggactacagcgacagcaccaaccccggaccccccaagaccccccctatcgccgtgcagaagatcagccagtacgcctgccagcggcggaccaccctgaacaactgcaaccagattttcaccgacgccttcgacatcctggccgaaaactgcgagttccgggagaacgaggacagctgcgtgaccttcatgagagccgccagcgtgctgaagtccctgcccttcaccatcatcagcatgaaggacaccgagggcatcccttgcctgggcagcaaagtgaagggcatcatcgaggaaatcattgaggacggcgagagcagcgaagtgaaagccgtgctgaacgacgagagataccagagcttcaagctgttcaccagcgtgttcggcgtgggcctgaaaaccagcgagaagtggttccggatgggcttcagaaccctgagcaaagtgcggagcgacaagagccttaagttcacccggatgcagaaggccggcttcctgtactacgaagatctggtgtcctgcgtgaccagagccgaggccgaggccgtgagcgtgctggtgaaagaggccgtctgggccttcctgcccgatgccttcgtgaccatgaccggcggcttcagacggggcaagaaaatgggccacgacgtggactttctgatcaccagccccggcagcaccgaggacgaagaacagctgctgcagaaagtgatgaacctgtgggagaagaagggcctgctgctgtactatgacctggtggagagcaccttcgagaagctgcggctgcccagccggaaggtggacgccctggaccacttccagaagtgctttctgatcttcaagctgcctcggcagagagtggacagcgaccagagcagctggcaggaaggaaagacctggaaggccatcagagtggacctggtgctgtgcccctacgagcggagagccttcgccctgctgggctggaccggcagccggcagttcgagcgggacctgcggagatacgccacccacgagcggaagatgatcctggacaaccacgccctgtacgacaagaccaagcggatcttcctgaaggccgagagcgaggaagaaatcttcgcccacctgggcctggactacatcgagccctgggagcggaacgcctaatctagagagagtttcagctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgggtaccgctagcgaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 41 bp, Artificial sequence
  RT primer
gagagtttggatcccaactttcttgtccaccttggtgttgc

SEQ ID NO: 4 - DNA, 50 bp, Artificial sequence
  AL63 primer sequence
gagagatttggtctctatgtcgatctgatcaagagacaggataaggagcc

SEQ ID NO: 5 - DNA, 38 bp, Artificial sequence
  AL891 primer sequence
gagagatttggtctcagctccacgtttttcaggtcctc

SEQ ID NO: 6 - DNA, 490 bp, Artificial sequence
  Representative amplicon sequences amplified using AL63 and AL891
gagagatttggtctctatgtcgatctgatcaagagacaggataaggagccgccaccatggagtttgggctgagctggctttttcttgtggctattttaaaaggtgtccagtgtgatgccgagatcacccagagccccagacacaagatcaccgagacaggtcgacaagtgaccctggcctgccaccagacctggaaccacaacaacatgttctggtacagacaggacctgggccacggcctgcggctgatccactactcttacggcgtgcaggacaccaacaagggcgaggtgtccgacggctacagcgtgtccagaagcaacaccgaggacctgcccctgaccctggaatctgccgccagcagccagaccagcgtgtacttctgcgccagcagtgagtggactagcggggatgagcagttcttcgggccagggacacggctcaccgtgctagaggacctgaaaaacgtggagctgagaccaaatctctc

SEQ ID NO: 7 - DNA, 5812 bp, Artificial sequence
  Surface expressed Fc-acceptor vector C857
tgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacacaagctggcggccgctctcggaaggtctctatgttgatcctttttaaccggtctcagagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaccggggagtcccttttaggcacttgcttctggtgctgcaactggcgctcctcccagcagccactcagggaaagaaagtggtgctgggcaacagcggcgattacaaggatgacgacgataaagttcggacgggaggtggcgggggttctaattccggagactacaaagacgatgatgacaaagtgggcggaggcgggagcctggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcg

SEQ ID NO: 8 - DNA, 490 bp, artificial sequence; identical to SEQ ID NO: 6
  amplicon

(19) United States
(12) Patent Application Publication
(10) Pub. No.: US 2019/0255186 A1
(43) Pub. Date: Aug. 22, 2019

(54) SINGLE VARIABLE DOMAIN T-CELL RECEPTORS

(71) Applicant: INNOVATIVE TARGETING SOLUTIONS INC., British Columbia (CA)

(72) Inventors: Michael Lajos GALLO, North Vancouver, British Columbia (CA); Jaspal Singh KANG, Surrey, British Columbia (CA); Craig Robin PIGOTT, Vancouver, British Columbia (CA); Abby LIN, Surrey, British Columbia (CA); Falene CHAI, Burnaby, British Columbia (CA)

(21) Appl. No.: 15/780,253

(22) PCT Filed: Dec. 2, 2016
(86) PCT No.: PCT/CA2016/051421

§ 371 Date: May 31, 2018

Related U.S. Application Data
(60) Provisional application No. 62/262,305, filed on Dec. 2, 2015.

(51) Int. Cl. A61K 47/66; A61K 47/68; A61P 35/00; A61K 51/08; C07K 14/725; G01N 33/567; C07K 14/74; C07K 16/28

(52) U.S. Cl. CPC A61K 47/66; A61K 47/68; A61P 35/00; C07K 16/2833; C07K 14/7051; G01N 33/567; C07K 14/70539; A61K 51/08

(57) ABSTRACT

There is provided a single variable domain T-cell receptor (svd-TCR) comprising a first TCR variable domain, the first TCR variable domain specifically binding to an epitope, that is not a superantigen, in the absence of a second TCR variable domain. Also provided are compositions and cells comprising the svd-TCR as well as methods of identifying the svd-TCR.

### FIELD OF INVENTION

The present invention relates to single variable domain T-cell receptors (svd-TCRs). More particularly, this disclosure relates to svd-TCRs which specifically bind to an epitope that is not a superantigen.

### BACKGROUND OF THE INVENTION

The emergence of biologics as useful medicines has in large part been due to the advancement of fully human monoclonal antibodies. Antibodies are the products of B-cells and are a central part of the body's humoral immune defense system. The primary function of antibodies is to bind to foreign antigens. V(D)J recombination, which occurs in the bone marrow, is a novel mechanism that generates B-cells which each produce a unique antibody. This somatic recombination event allows for large repertoires of antibodies to be generated as part of an animal's defense against pathogens. Antibodies can have high affinities, nanomolar or even subnanomolar, a result of affinity maturation that occurs in secondary immune organs such as the spleen and lymph nodes. Antibodies bind to secreted and membrane expressed antigens. Although antibodies have been generated to a wide variety of antigens and would seem to have the potential to bind to any target, some targets have historically been a challenge. Despite a variety of technologies, including transgenic animals, phage display, yeast display and ribosome display as examples, one class of target that is historically difficult to routinely generate antibodies against a peptide bound in a major histocompatibility complex (MHC), i.e. MHC:peptide complexes (pMHCs).

The MHC is a two chained cell surface protein that binds to proteolytic fragments (i.e. peptides) of proteins expressed by a given cell. As a result, a given cell “presents” on its cell surface, a composite of its expressed proteins. Thus, if a cell is infected with a virus or parasite, for example, these proteins are also externally presented on the cell surface.

Although the literature describes a handful of examples, the ability to generate antibodies that recognize a specific pMHC while not binding to the MHC molecule in the absence of peptide or in the presence of another irrelevant peptide is nevertheless challenging. The challenge resides in the fact that there are significantly more potential epitopes represented by the surface of MHC compared to the restricted epitope surface of the specific peptide fragment, which may be as short as approximately 8-10 amino acids. When immunizing an animal or panning with phage display, the majority of all binders will thus not be to the desired epitope.

In addition to the humoral immune system, animals also have a cellular immune system that includes T-cells. T-cell receptors (TCRs) are also generated by V(D)J recombination, the same system that generates B-cell repertoires, resulting in variable regions comprising six complementarity determining regions (CDRs). TCRs, however, are generated in the thymus and specifically recognize pMHCs. Unlike antibodies, TCRs do not undergo affinity maturation in vivo and generally do not have subnanomolar affinities unless they are recombinantly engineered. In fact, it is hypothesized that there is a narrow affinity range appropriate for a TCR-pMHC interaction. In vivo, tolerance in the thymus deletes T-cells that have TCRs with high intrinsic affinity to MHC to eliminate binding in a peptide independent manner. Likewise, T-cells that have TCRs with too low an affinity are deleted from the repertoire.

It is well appreciated that the ability to specifically bind to pMHCs would provide novel therapeutic potential since it would allow the targeting of cells based on the expression of their intracellular proteins. Appreciating the challenges of using antibodies to generate binding modalities with this fine specificity, the field is now beginning to explore the use of TCRs. The TCR is comprised of two chains, either a beta chain and an alpha chain or a delta chain and gamma chain, in which all chains contain transmembrane sequences that result in their being surface expressed. TCRs, unlike antibodies, are not secreted. The two chains are membrane expressed proteins that are assembled as part of a larger protein complex, the T-cell receptor complex, which includes CD3. Alpha:beta (α:β) or delta:gamma (δ:γ) chain association outside of the TCR complex is not very efficient or stable. Although there are reports of single-chain molecules, these molecules recapitulate the heterodimeric binding site and incorporate both an alpha chain variable sequence and a beta chain variable sequence into a single molecule. The association of the two domains outside of the complete TCR complex expressed on the cell surface has required modifications to stabilize the interaction and has included relatively long linker sequences between the beta and alpha TCR sequences to generate a single recombinant molecule.

There have been no reports of TCRs which do not contain a heterodimeric binding domain since using a single chain or variable domain of the TCR to target pMHCs was not considered to be an option. By comparison, heavy chain only antibodies (HCAbs) have been shown to exist in dromedaries, camels, llamas, alpacas but not in other mammals. HCAbs have also been reported in sharks but evolutionary analysis indicate that camelid and shark HCABs evolved independently.

Peptides bind MHC in a distinctive cleft or groove. Class I MHCs typically bind peptides which are 8-10 amino acids long. Class II MHCs typically bind peptides which are 8-30 amino acids long, although they may be longer.

Superantigens (SAgs) also bind both the human TCR and the MHC. SAgs are a class of antigen that cause non-specific activation of T-cells resulting in polyclonal T-cell activation and massive cytokine release. They are produced by some pathogenic viruses and bacteria, potentially as a defense mechanism against the immune system. Binding of SAgs to the TCR is by a different mechanism than classic TCR recognition of pMHCs and is independent of the TCR CDR3 sequences of the variable domains. This allows SAgs to cause broad (non-specific) T-cell activation. SAgs have multiple domains that act to bridge the TCR and MHC complex. SAgs produced intracellularly by bacteria are relatively conserved. Cyrstal structures of the enterotoxins reveal that they share a characteristic two-domain folding pattern comprising an amino terminal β-barrel globular domain 1, a long α-helix and a carboxy terminal globular domain II. The domains have binding regions for the Class II MHC and the TCR, respectively. Certain Group I SAgs contact the Vβ at the CDR2 and framework regions of the TCR. Certain SAgs of Group II interact with the Vβ region using mechanisms that are conformation dependent. These interactions are for the most part independent of specific Vβ amino acid side-chains. Certain Group IV SAgs have been shown to engage all three CDR loops of certain Vβ forms. The interaction takes place in a cleft between the small and large domains of the SAg and allows the SAg to act as a wedge between the TCR and MHC. This displaces the antigenic peptide away from the TCR and circumvents the normal mechanism for T-cell activation. SAgs appear to cross-link the MHC and the TCR, inducing a signaling pathway. Accordingly, a given SAg can activate a large proportion of the T-cell population because the human T-cell repertoire comprises only about 50 types of Vβ elements and some SAgs are capable of binding to multiple types of Vβ regions. Although SAgs simultaneously interact with TCRs and MHCs, their interaction is distinct from the interaction of normal T-cells and pMHCs. The latter are dependent on specific peptides presented in the MHC and specific CDR sequences in the TCR that mediate the recognition of the peptide sequences in the context of MHC. This fine specificity allows for differentiation of self from non-self and is in direct contrast to the polyclonal activation mediated by SAgs. The normal TCR-pMHC interaction allows for the activation of specific T-cells that recognize peptides derived from the expression of intracellular proteins and do not inherently activate TCRs in a polyclonal manner like SAgs.

No admission is necessarily intended, nor should it be construed, that any of the preceding information constitutes prior art against the present invention.

### SUMMARY OF THE INVENTION

The present invention relates to single variable domain T-cell receptors (svd-TCRs).

Various embodiments of the present disclosure relate to a single variable domain T-cell receptor (svd-TCR) comprising a first TCR variable domain, the first TCR variable domain specifically binding to an epitope in the absence of a second TCR variable domain, wherein the epitope is not a superantigen. The first TCR variable domain may comprise a TCR Vα domain, a TCR Vβ domain, a TCR Vγ domain or a TCR Vδ domain. The svd-TCR may comprise a TCR α chain, a TCR β chain, a TCR γ chain or a TCR δ chain. The first TCR variable domain may be a human TCR variable domain.

The epitope may be a peptide bound in a major histocompatibility complex (MHC) to form a MHC:peptide complex (pMHC). The MHC may be a class I MHC or a class II MHC.

The svd-TCR may be fused to an antibody Fc (svd-TCR-Fc).

The svd-TCR may be fused to a membrane anchor (svd-TCR-anchor).

The svd-TCR be part of a soluble fusion protein. The soluble fusion protein may comprise: an anticancer agent; a therapeutic radionuclide; a cytotoxic protein; a marker; or a combination thereof.

The svd-TCR be part of a chimeric antigen receptor (svd-TCR-CAR).

Various embodiments of the present disclosure relate to a composition comprising the svd-TCR, svd-TCR-Fc, svd-TCR-anchor, svd-TCR-CAR or fusion protein defined above, and a pharmaceutically acceptable excipient.

Various embodiments of the present disclosure relate to a cell comprising a nucleic acid sequence encoding the svd-TCR defined above, wherein the nucleic acid sequence is in operative association with a promoter and terminator for expression of the svd-TCR. The cell may be a human T-cell or NK cell. Various embodiments of the relate to a composition comprising the cell and a pharmaceutically acceptable excipient.

Various embodiments of the present disclosure relate to a method of identifying a svd-TCR which specifically binds to a peptide bound in a MHC (pMHC), the svd-TCR comprising a first TCR variable domain which specifically binds to the peptide in the absence of a second TCR variable domain. The method comprises: providing a pool of eukaryotic cells which externally present a plurality of unique svd-TCRs that have different variable domains; contacting the pool of eukaryotic cells with two different pMHCs, wherein the two different pMHCs have the same major histocompatibility complex (MHC) but different peptides, one of the different peptides being the desired peptide, and wherein each of the two different pMHCs is labeled with a distinguishable marker; identifying a cell that binds to one of the two different pMHCs and does not bind to the other of the two different pMHCs; and identifying from the identified cell the svd-TCR which specifically binds the desired peptide in the pMHC. The plurality of unique TCR variable domains may comprise at least 10 million unique TCR variable domains. Said identifying may comprise isolating from the identified cell the svd-TCR which specifically binds the desired peptide in the pMHC. The method may further comprise producing the pool of eukaryotic cells using in vitro V(D)J recombination.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a vector diagram for P273.

FIG. 2 shows a vector diagram for P262.

FIG. 3 shows the sequence of P273.

FIG. 4 shows the sequence of P262.

FIG. 5 shows a FACS plot of HEK293 cells surface displaying fully human antibodies and stained for binding to two MHC:peptide complexes (50,000 cells displayed). MHC:NY-ESO; MHC A*02:01 1 μg/mL with Avidin-PE 1 μg/mL. MHC:HIV; MHC A*02:01 1 μg/ml with Avidin-647 1 μg/mL. The x-axis is the geomean of PE fluorescence observed on cells resulting from the surface displayed antibody binding to the NY-ESO peptide containing complex. The y-axis is the geomean of Alexa Fluor™-647 fluorescence observed on cells resulting from the surface displayed antibody binding to the HW peptide containing complex.

FIG. 6 shows a FACS plot of HEK293 cells surface displaying fully human T-cell receptors and stained for binding to two MHC:peptide complexes (1000 cells displayed). MHC:NY-ESO; MHC A*02:01 1 μg/mL with Avidin-PE 1 μg/mL. MHC:HIV; MHC A*02:01 1 μg/mL with Avidin-647 1 μg/mL. The x-axis is the geomean of PE fluorescence observed on cells resulting from the surface displayed antibody binding to the NY-ESO peptide containing complex. The y-axis is the geomean of Alexa Fluor™-647 fluorescence observed on cells resulting from the surface displayed antibody binding to the HW peptide containing complex

FIG. 7 shows a FACS plot of HEK293 cells surface displaying recombinant svd-TCRs and stained for binding to MHC:peptide complexes. Recombinant svd-TCRs were transiently transfected and then incubated with an anti-TCR beta antibody labeled with PE and an Alexa Fluor™-647 labeled complex. MHC:NY-ESO; MHC A*02:01 1 μg/ml with Avidin-647 1 μg/ml. MHC:HIV; MHC A*02:01 1 μg/mL with Avidin-647 1 μg/mL. The x-axis the geomean reflecting the amount of TCR beta on the surface of the cell. The y-axis is the geomean flouresence resulting from the binding to the Alexa Fluor™-647 labeled MHC complex. In the top panel the cells were stained with pMHC containing the NY-ESO peptide and the bottom panel the cells were stained with pMHC containing a HIV peptide.

FIG. 8. FIG. 8A shows the nucleic acid sequence of primers AL63 (SEQ ID NO: 4) and AL891 (SEQ ID NO: 5). FIG. 8B shows the nucleic acid sequence of a representative PCR product (SEQ ID NO: 6) using AL63 and AL891. FIG. 8C shows a plasmid map of vector C857. FIG. 8D shows the nucleic acid sequence of vector C857. FIG. 8E shows the nucleotide sequence of an example amplicon cloned into C857 with BSAI compatible overhangs (BSAI restriction sites are underlined in the forward primer and reverse primer sequences; AL63 primer residues 1-50; Kozak residues 48-59; IGHV3-23 Leader residues 57-113; TRBV10-1*01 residues 114-399; TRBD2*01 residues 400-411; TRBJ2-1*01 residues 412-452; TCR Beta-2 Constant ECD residues 453-470; AL891 (rev primer) residues 490-453). FIG. 8F shows a FACS plot of HEK293 cells transfected with pUC19 (negative control; left column), HEK293 cells surface displaying a fully human antibody (Fc-positive control; middle column) or a pool of HEK293 cells surface displaying svd-TCR Fc-fusion proteins. Cells were stained for Fc-binding using a fluorescent goat anti-human-Fc-PE conjugated polyclonal antibody (horizontal axis), and stained for binding to biotinylated MHC:peptide complexes (vertical axis) using MHC:A*02:01 MAGE-A3 (bottom row) or MHC:A*02:01 PSA-1 (top row). Biotinylated MHC/peptide complexes were detected with Alexa Fluor™-647 streptavidin.

### DETAILED DESCRIPTION

### Definitions

As used herein, the terms “comprising,” “having”, “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the content clearly dictates otherwise. The use of the word “a” or “an” when used herein in conjunction with the term “comprising” may mean “one,” but it is also consistent with the meaning of “one or more,” “at least one” and “one or more than one.”

Unless indicated to be further limited, the term “plurality” as used herein means more than one, for example, two or more, three or more, four or more, and the like.

As used herein, the term “about” refers to an approximately +/−10% variation from a given value. It is to be understood that such a variation is always included in any given value provided herein, whether or not it is specifically referred to.

In this disclosure, the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, protein, nucleic acid, at least one nucleic acid, cell, cell, kit, et cetera.

As used herein, a “polypeptide” is a chain of amino acid residues, including peptides and protein chains. A polypeptide may include amino acid polymers in which one or more of the amino acid residues is an artificial chemical analogue of a corresponding naturally occurring amino acid, or is a completely artificial amino acid with no obvious natural analogue as well as to naturally occurring amino acid polymers.

As used herein, “nucleic acid”, “nucleic acid sequence”, “nucleotide sequence”, or similar terms mean oligomers of bases typically linked by a sugar-phosphate backbone, such as oligonucleotides or polynucleotides, and to DNA or RNA of genomic or synthetic origin which can be single-or double-stranded, and represent a sense or antisense strand. The terms nucleic acid, polynucleotide and nucleotide also specifically include nucleic acids composed of bases other than the five biologically occurring bases (i.e., adenine, guanine, thymine, cytosine and uracil), and also include nucleic acids having non-natural backbone structures. Unless otherwise indicated, a particular nucleic acid sequence of this invention encompasses complementary sequences, in addition to the sequence explicitly indicated.

In this disclosure, “nucleic acid vector”, “vector” and similar terms refer to at least one of a plasmid, bacteriophage, cosmid, artificial chromosome, expression vector, or any other nucleic acid vector. Those skilled in the art, in light of the teachings of this disclosure, will understand that alternative vectors may be used, or that the above vectors may be modified in order to combine sequences as desired. For example, vectors may be modified by inserting additional origins of replication, or replacing origins ofreplication, introducing expression cassettes comprising suitable promoter and termination sequences, adding one or more than one DNA binding sequence, DNA recognition site, or adding sequences encoding polypeptides as described herein, other products of interest, polypeptides of interest or proteins of interest, or a combination thereof. In some embodiments adjacent functional components of a vector may be joined by linking sequences.

A “coding sequence” or as sequence which is “encoded”, as used herein, includes a nucleotide sequence encoding a product of interest, for example a peptide or polypeptide, or a sequence which encodes RNA that lacks a translation start and/or stop codon or is otherwise unsuitable for translation into a peptide or polypeptide, for example, an RNA precursor of small interfering RNAs (siRNAs) or microRNAs (miRNAs).

A “promoter” is a DNA region, typically but not exclusively 5′ of the site of transcription initiation, sufficient to confer accurate transcription initiation. The promoter nucleic acid typically contains regions of DNA that are involved in recognition and binding of RNA polymerase and other proteins or factors to initiate transcription. In some embodiments, a promoter is constitutively active, while in alternative embodiments, the promoter is conditionally active (e.g., where transcription is initiated only under certain physiological conditions). Conditionally active promoters may thus be “inducible” in the sense that expression of the coding sequence can be controlled by altering the physiological condition.

A “terminator” or “transcription termination site” refers to a 3′ flanking region of a gene or coding sequence that contains nucleotide sequences which regulate transcription termination and typically confer RNA stability.

As used herein, “operably linked”, “operatively linked”, “operative association” and similar phrases, when used in reference to nucleic acids, refer to the linkage of nucleic acid sequences placed in functional relationships with each other. For example, an operatively linked promoter sequence, open reading frame and terminator sequence results in the accurate production of an RNA molecule. In some aspects, operatively linked nucleic acid elements result in the transcription of an open reading frame and ultimately the production of a polypeptide (i.e., expression of the open reading frame).

With respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977 (J. Pharm Sci. 66:1-19), or Remington—The Science and Practice of Pharmacy, 21st edition (Gennaro et al editors. Lippincott Williams & Wilkins Philadelphia), both of which are herein incorporated by reference.

Certain embodiments of the present disclosure relate to a monomeric T-cell receptor comprising a first TCR variable domain, the first TCR variable domain specifically binding to an epitope, that is not a superantigen, in the absence of a second TCR variable domain. As used herein, superantigen (or SAg) are antigens that cause non-specific T-cell activation and are further defined in the Background section of this application.

Certain embodiments of the present disclosure relate to a pMHC-binding molecule comprising a first TCR variable domain, the first TCR variable domain specifically binding to the pMHC in the absence of a second TCR variable domain. The term “pMHC” refers to a peptide bound in a MHC as a MHC:peptide complex. As such, specific binding of the pMHC is distinct from binding the MHC in the absence of the peptide or a complex of the MHC bound to a different peptide.

Certain embodiments of present disclosure relate to a single variable domain T-cell receptor (svd-TCR) comprising a first TCR variable domain, the first TCR variable domain specifically binding to an epitope in the absence of a second TCR variable domain, wherein the epitope is not a superantigen. Unless otherwise indicated, a svd-TCR may include additional elements besides the first TCR variable domain, including additional amino acid sequences, additional protein domains (covalently associated, non-covalently associated or covalently and non-covalently associated with the TCR variable domain), fusion or non-covalent association of the TCR variable domain with other types of macromolecules (for example polynucleotides, polysaccharides, lipids, or a combination thereof), fusion or non-covalent association of the TCR variable domain with one or more small molecules, compounds, or ligands, or a combination thereof. Any additional element, as described, may be combined provided that the first TCR variable domain is configured to specifically bind the epitope in the absence of a second TCR variable domain.

An svd-TCR as described herein may comprise a single TCR chain (e.g. α, β, β, or δ chain), or it may comprise a single TCR variable domain (e.g. of α, β, γ, or δ chain). If the svd-TCR is a single TCR chain, then the TCR chain comprises a transmembrane domain, a constant (or C domain) and a variable (or V domain), and does not comprise a second TCR variable domain. The svd-TCR may therefore comprise or consist of a TCR α chain, a TCR β chain, a TCR γ chain or a TCR δ chain. The svd-TCR may be a membrane bound protein. The svd-TCR may alternatively be a membrane-associated protein.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347:166). Non-limiting examples of membrane anchor systems include platelet derived growth factor receptor (PDGFR) transmembrane domain, glycosylphosphatidylinositol (GPI) anchor (added post-translationally to a signal sequence) and the like.

It is known in the art that the TCR variable domain can be stably expressed without the TCR transmembrane domain or TCR C domain to generate a soluble protein (Alajez et. al. 2006 Journal of BioMedicine and Biotechnology 2006:1-9; Laugel et al. 2005 J. Biol. Chem. 280:1882-1892). Thus, the svd-TCR as described herein may be a soluble protein. For example, the svd-TCR may comprise a single TCR variable domain (i.e. the first TCR variable domain) without the transmembrane or C domains (or portions thereof). The first TCR variable domain may comprise either a TCR Vα domain, a TCR Vβ domain, a TCR Vγ domain, or a TCR Vδ domain. Therefore, the soluble svd-TCR may comprise the first TCR variable domain, for example, a single TCR variable α, β, γ or δ domain.

The first TCR variable domain may be a human TCR variable domain. Alternatively, the first TCR variable domain may be a non-human TCR variable domain. The first TCR variable domain may be a mammalian TCR variable domain. The first TCR variable domain may be a vertebrate TCR variable domain.

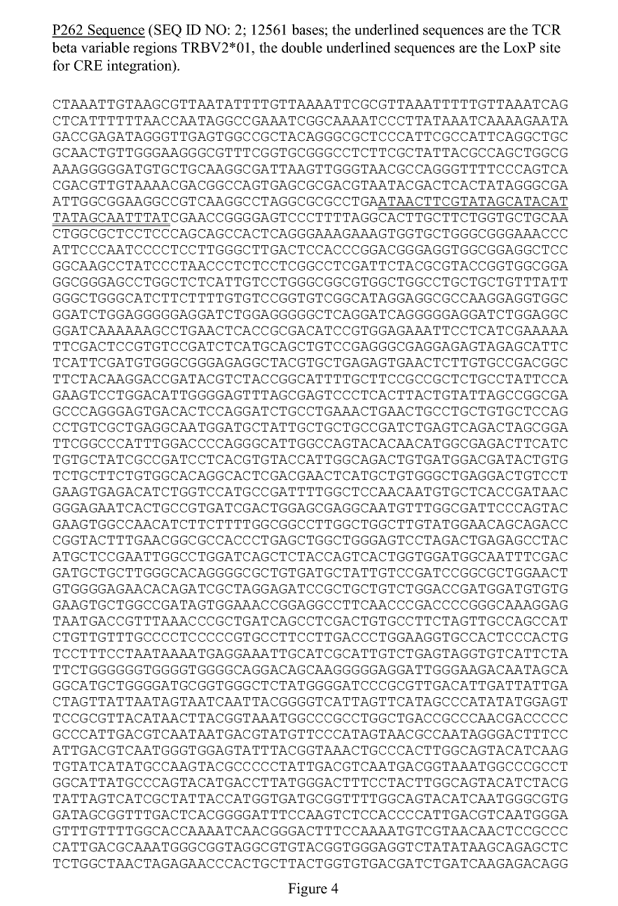

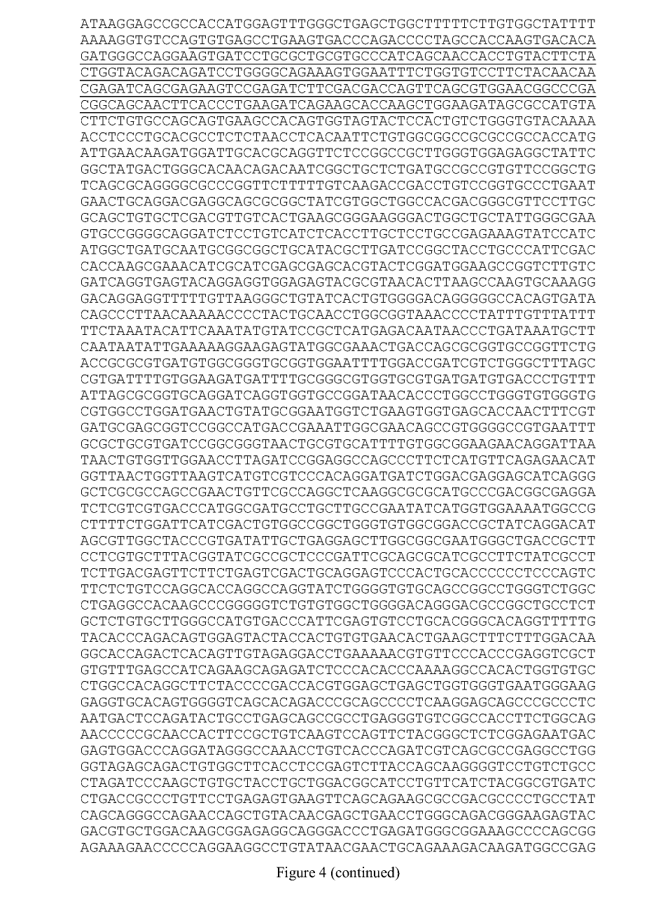

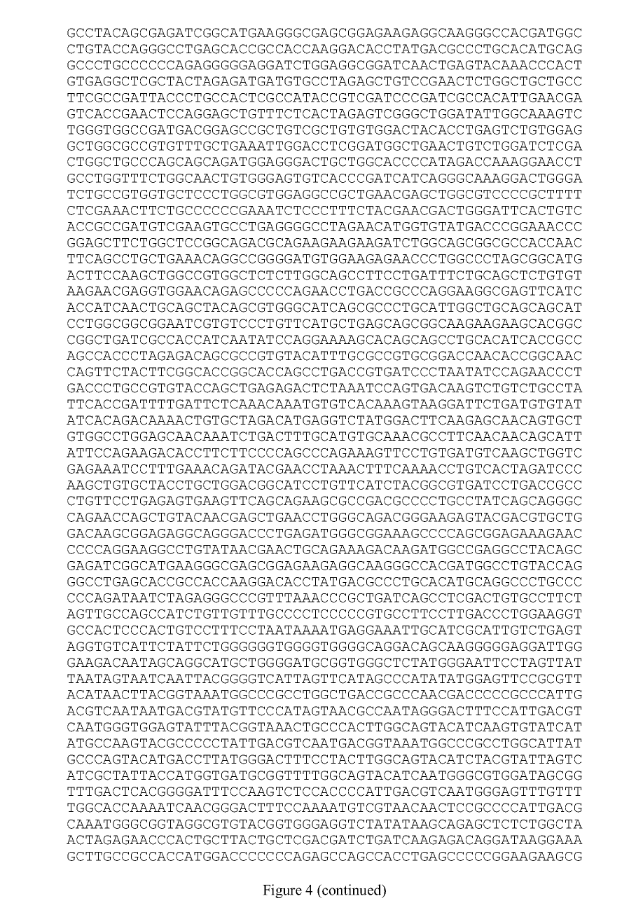

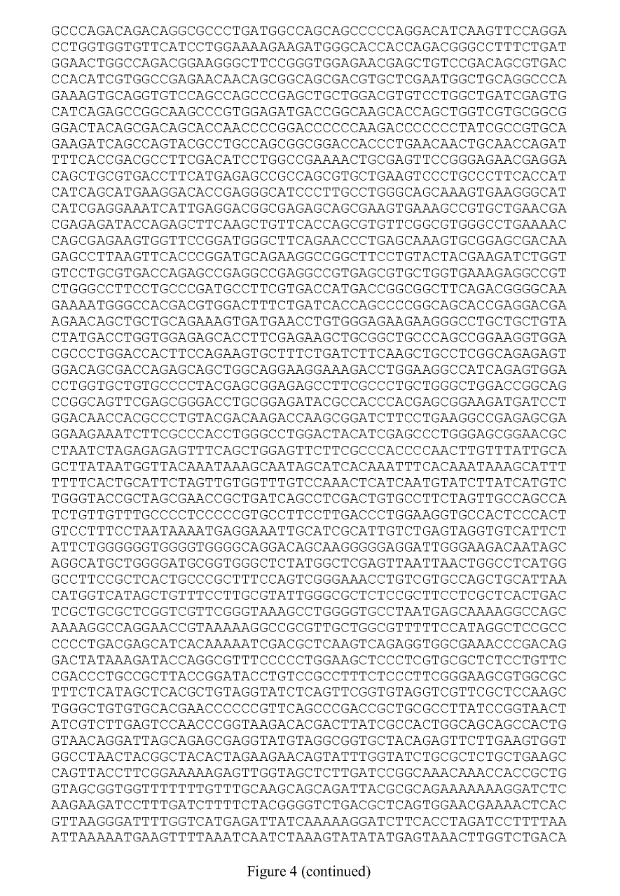

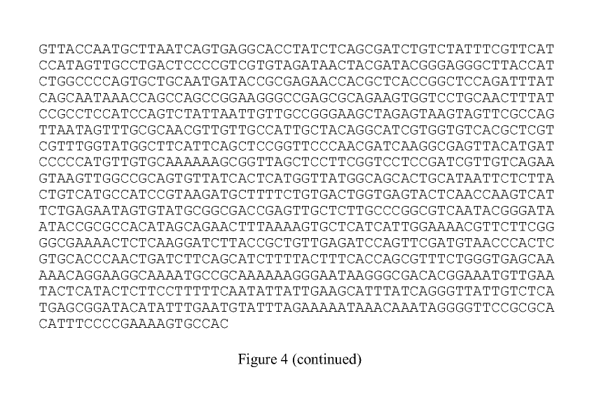

In humans, the TCR variable regions of the α and γ chains are each encoded by a V and a J segment, whereas the variable region of β and δ chains are each additionally encoded by a D segment. There are multiple Variable (V), Diversity (D) and Joining (J) gene segments (e.g. 52 Vβ gene segments, 2 Dβ gene segments and 13 Jβ gene segments) (Janeway et al. (eds.), 2001, Immunobiology: The Immune System in Health and Disease. 5^{th} Edition, New York, FIG. 4.13) which can be recombined in different V(D)J arrangements using the enzymes RAG-1 and RAG-2, which recognize recombination signal sequences (RSSs) adjacent to the coding sequences of the V, D and J gene segments. The RSSs consist of conserved heptamers and nonamers separated by spacers of 12 or 23 bp. The RSSs are found at the 3′ side of each V segment, on both the 5′ and 3′ sides of each D segment, and at the 5′ of each J segment. During recombination, RAG-1 and RAG-2 cause the formation of DNA hairpins at the coding ends of the joint (the coding joint) and removal of the RSSs and intervening sequence between them (the signal joint). The variable regions are further diversified at the junctions by deletion of a variable number of coding end nucleotides, the random addition of nucleotides by terminal deoxynucleotidyl transferase (TdT), and palindromic nucleotides that arise due to template-mediated fill-in of the asymmetrically cleaved coding hairpins.

Patent application WO 2009/129247 (herein incorporated by reference in its entirety) discloses an in vitro system utilizing V(D)J recombination to generate de novo antibodies in vitro. This same system may be used to generate the variable regions of the svd-TCR as described herein by using TCR-specific V, D and J elements. In natural in vivo systems, the nucleic acid sequences which encode CDR1 and CDR2 are contained within the V (α, β, γ or δ) gene segment and the sequence encoding CDR3 is made up from portions of V and J segments (for Vα or Vγ), or a portion of the V segment, the entire D segment and a portion of the J segment (for Vβ or Vδ), but with random insertions and deletions of nucleotides at the V-J and V-D-J recombination junctions due to action of TdT and other recombination and DNA repair enzymes. The recombined T-cell receptor gene comprises alternating framework (FR) and CDR sequences, as does the resulting T-cell receptor expressed therefrom (i.e. FR1-CDR1-FR2-CDR2-FR3-CDR3-FR4). Using in vitro V(D)J recombination (i.e. V-J or V-D-J recombination), randomized insertions and deletions may be added in or adjacent to CDR1, CDR2 and/or CDR3 (i.e. not just CDR3), additional insertions may be added using flanking sequences in recombination substrates before and/or after CDR1, CDR2 and/or CDR3, and additional deletions may be made by deleting sequences in recombination substrates in or adjacent to CDR1, CDR2 and/or CDR3.

In some embodiments, the first TCR variable domain of the svd-TCR specifically binds to an epitope in the absence of a second TCR variable domain, wherein the epitope is not a superantigen, and consists of optional N-terminal and/or C-terminal amino acid sequences (of any length or sequence) flanking a variable domain defined by FR1-CDR1-FR2-CDR2-FR3-CDR3-FR4 regions. FR1, FR2, FR3 and FR4 may be obtained from a natural Vα, Vβ, Vγ or Vδ domain or encoded by natural Vα, Vβ, Vγ or Vδ gene segments, but optionally include deletions or insertions of (e.g. 0, 1, 2, 3, 4, 5 or more than 5 amino acids) amino acids independently at one or more of the C-terminus of FR1, the N-terminus of FR2, the C-terminus of FR2, the N-terminus of FR3, the C-terminus of FR3 and the N-terminus of FR4. CDR1, CDR2 and CDR3 may be obtained from a natural Vα, Vβ, Vγ or Vδ domain, or encoded by natural Vα, Vβ, Vγ or Vδ gene segments, but wherein one or more of CDR1, CDR2 and CDR3 independently contains an insertion (e.g. 0, 1, 2, 3, 4, 5, 6, 7, 8, 9, 10 or more than 10 amino acids) and/or a deletion (e.g. 0, 1, 2, 3, 4, 5 or more than 5 amino acids) at the C-terminus, the N-terminus or anywhere within the CDR sequence. In some embodiments, the CDR1 contains an insertion or deletion of amino acids N-terminally, C-terminally or internally, wherein at least 50% (or optionally 60%, 70% or 80%) of natural CDR amino acid residues are retained. In some embodiments, the CDR2 contains an insertion or deletion of amino acids N-terminally, C-terminally or internally, wherein at least 50% (or optionally 60%, 70% or 80%) of natural CDR amino acid residues are retained. In some embodiments, the CDR3 contains an insertion or deletion of amino acids N-terminally, C-terminally or internally, wherein at least 50% (or optionally 60%, 70% or 80%) of natural CDR amino acid residues are retained. Insertions and/or deletions may be produced as a result of in vitro V(D)J recombination methods or from the in-vitro action of TdT and recombination and DNA repair enzymes (e.g. one or more of Artemis nuclease, NDA-dependent protein kinase (DNA-PK), X-ray repair cross-complementing protein 4 (XRCC4), DNA ligase IV, non-homologous end joining factor 1 (NHEJ1), PAXX, and DNA polymerases λ and μ). Insertion and/or deletion (which includes substitution) may further result from insertions and/or deletions to CDR nucleic acid sequences of the in vitro V(D)J recombination substrates. The svd-TCR may further comprise a TCR constant region or portion thereof. The svd-TCR may be fused to and/or complexed with additional protein domains. A double stranded break in DNA may be introduced prior to in vitro use of the above recombination and DNA repair enzymes.

The epitope (or epitope of interest) which is specifically bound by the TCR variable domain of the svd-TCR may be any epitope. The epitope may be a self epitope or a non-self epitope. The epitope may be a conformational epitope or a linear epitope. Non-limiting examples of the epitope include viral proteins or peptides, bacterial proteins or peptides, cancer-specific epitopes, receptor extracellular domains, an antigen, a receptor binding protein, a receptor binding peptide, or any other peptide, polypeptide or protein epitope.

In some embodiments, the epitope is a peptide bound in a major histocompatibility complex (MHC) to form a pMHC. The MHC in the pMHC may be any MHC class. For example, the MHC may be MHC class I or may be MHC class II. The peptide in the pMHC may be any length that will bind in the binding groove of the MHC. For example, the peptide may be 2 to 100 or more amino acids long, including a peptide of 2, 3, 4, 5, 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 54, 56, 68, 60, 62, 64, 66, 68, 70, 75, 80, 85, 90, 95, 100 amino acids. The peptide may be about 8-30 amino acids long. The peptide may be about 8-10 amino acids long. The peptide in the pMHC may be any sequence that binds in the binding groove of the MHC. The peptides are non-covalently held in the binding groove in an extended configuration but some peptides may have portions which dangle outside the binding groove. In a non-limiting example, the peptide may have the amino acid sequence SLLMWITQC (a publicly known sequence).

The svd-TCR may be (or may be incorporated into) a fusion protein. As used herein, the term “fusion protein” means a protein encoded by at least one nucleic acid coding sequence that is comprised of a fusion of two or more coding sequences from separate genes, regardless of whether the organism source of those genes is the same or different.

In certain embodiments, the svd-TCR fusion protein may comprise an agent of interest or comprise a binding domain for non-covalent association with, or covalent attachment to, an agent of interest. For example, but without limitation, a single TCR chain may be fused to an agent of interest, or a single TCR variable domain may be fused to an agent of interest. Without limitation, the svd-TCR fusion may comprise: a diagnostic agent; an anticancer agent; a therapeutic radionuclide; a cytotoxic protein; a marker; a purification tag; an epitope; a ligand; a membrane anchor; or a combination thereof. Non-limiting examples of diagnostic agents or moieties include radioisotopes and other detectable labels. Detectable labels useful for such purposes are well known in the art, and include radioactive isotopes such as ³²P, ¹²⁵I, and ¹³¹I, fluorophores, chemiluminescent agents, and enzymes. Non-limiting examples of cytotoxic proteins comprise toxins such as abrin, ricin, Pseudomonas exotoxin (PE; such as PE35, PE37, PE38, and PE40), diphtheria toxin (DT) and subunits thereof, botulinum toxin (e.g. botulinum toxin A through F), or modified toxins thereof, or other toxic agents that directly or indirectly inhibit cell growth or kill cells as well as other proteins that once internalized are toxic to the cell. Toxins can be fused to a svd-TCR for use as an immunotoxin. Non-limiting examples of markers comprise GFP (green fluorescent protein), RFP (red fluorescent protein), CAT (chloramphenicol acetyltransferase), luciferase, GAL (beta-galactosidase), GUS (beta-glucuronidase) and the like. Non-limiting examples of purification tags include peptide tags (e.g. FLAG, V5 and the like), polyhistidine tags, glutathione S-transferase (GST) tags, maltose binding protein (MBP) tags, calmodulin binding peptide tags, intein-chitin binding domains, streptavidin/biotin-based tags, and the like (see, e.g., Kimple et al., Overview of Affinity Tags for Protein Purification. Current protocols in protein science/editorial board, John E Coligan et al. 2013;73:Unit-9.9.doi:10.1002/0471140864.ps0909s73). In some embodiments, the svd-TCR may comprise or be fused to additional binding/association domain(s) (e.g. ligands, epitopes and the like). For example, the svd-TCR fusion may comprise bispecific or multispecific elements to recruit immune cells like NK or T-cells to the target cell. There are currently over 60 different bi-specific antibody formats that have been described in the literature (see, e.g., Spiess et al. Mol Immunol. 2015 67(2 Pt A):95-106). Multispecific formats may be generated by adding antibody or TCR V_{H} domains or other binding modalities to these scaffold or engineering in additional binding specificities into an antibody or TCR constant region.

In embodiments where the svd-TCR is incorporated into a fusion protein. The fusion protein may comprise a svd-TCR and any other protein domain or domains. In some embodiments, for example, but without limitation, the svd-TCR may be incorporated into an Fc-fusion (i.e. an svd-TCR-Fc) and still retain its binding properties of recognizing specific pMHC complexes. The Fc domain maybe N- or C-terminal to the svd-TCR portion. Among other advantages/uses, the svd-TCR-Fc fusion protein allows for a robust approach to generating soluble MHC/peptide binders. The svd-TCR-Fc may alternatively be membrane bound (e.g. cell surface displayed). In some embodiments, the Fc domain may provide extended half-life and/or ease of purification.

In some embodiments, the domains (e.g. heterologous or homologous domains) of the fusion protein may be fused via a linker (e.g. a peptide linker). Any linker may be used and many fusion protein linker formats are known. For example, the linker may be flexible or rigid. Non-limiting examples of rigid and flexible linkers are provided in Chen et al. (Adv Drug Deliv Rev. 2013; 65(10):1357-1369). In some embodiments, the linker is a peptide of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 amino acid residues. Non-limiting examples of amino acids found in linkers include Gly, Ser, Glu, Gln, Ala, Leu, Iso, Lys, Arg, Pro, and the like. In some embodiments, the linker is [(Gly)ₙ₁Ser]ₙ₂, where n1 and n2 may be any number (e.g. n1 and n2 may independently be 1, 2, 4, 5, 6, 7, 8, 9, 10 or more than 10). In some embodiments, n1 is 4.

The svd-TCR may be or may form part of a chimeric antigen receptor (CAR). A CAR is a recombinant fusion protein in which a binding domain, a transmembrane domain and a signaling domain or domains are linked to create a novel receptor. Typically antibody scFVs are used as the binding domain. A CAR may be created from a svd-TCR by linking a single TCR variable domain to transmembrane domain and signaling domain(s) or by linking a single TCR chain to a signaling domain or domains, for example.

Certain embodiments relate to a composition comprising the svd-TCR and a pharmaceutically acceptable excipient.

Also provided is at least one nucleic acid encoding the svd-TCR as defined herein. The at least one nucleic acid may be a vector. The vector may be an expression vector. The at least one nucleic acid may comprise an expression cassette comprising a sequence encoding the svd-TCR and further comprising a promoter and terminator in operative association with the sequence encoding the svd-TCR for expression ofthe svd-TCR. Non-limiting examples of promoters which may be suitable include, but are not limited to CMV, SV40, E1a, viral LTRs, heat shock promoters, viral and chimeric promoters, tetracycline or other inducible promoters. Non-limiting examples of terminators which may be suitable include, but are not limited to SV40 poly (A), bovine growth hormone poly(A) or synthetic poly (A) sequences.

Also provided is a cell comprising a nucleic acid encoding the svd-TCR. In some embodiments, the nucleic acid is in operative association with a promoter and terminator for expression of the svd-TCR. Non-limiting examples of mammalian promoters which may be suitable include, but are not limited to CMV, SV40, E1a, viral LTRs, heat shock promoters, viral and chimeric promoters, tetracycline or other inducible promoters. Non-limiting examples of terminators which may be suitable include, but are not limited to SV40 poly (A), bovine growth hormone poly(A) or synthetic poly (A) sequences. The cell may be a eukaryotic cell. The cell may be a vertebrate cell. The cell may be a mammalian cell. The cell may be a T-cell (e.g. a mammalian T-cell). The cell may be a human T-cell. The cell may be a non-human T-cell. Also provided is a composition comprising the cell and a pharmaceutically acceptable excipient.

Certain embodiments relate to use of the svd-TCR or the compositions disclosed herein for targeting a cell which externally presents an epitope of interest. Similarly, certain embodiments relate to a method of targeting a cell which externally presents an epitope of interest (e.g. a pMHC), comprising contacting the cell with the svd-TCR or a composition as disclosed herein. Any cell which expresses the epitope on its cell surface may be targeted, including, for example, cancer cells, autoreactive immune cells, or the like.

The targeting may be for diagnostic purposes, screening purposes, therapeutic purposes, or any other purpose. In a non-limiting example, svd-TCRs which are soluble fusion proteins comprising an agent of interest, may be used to target an anticancer agent, fused as part of the svd-TCR, to cells in a subject which express a cancer-specific cell surface epitope. In another non-limiting example, svd-TCRs which when formatted for use as soluble fusion proteins comprising an agent of interest, may be used to specifically target a cytotoxic protein, fused as part of the svd-TCR, to undesired cells in a subject which express a cell surface epitope specific for an undesired cell. For example, which is not to be considered limiting, the epitope may be a pMHC comprising proteolyzed fragments of bacterial or viral proteins which are being intracellularly expressed. In another non-limiting example, svd-TCRs which are soluble fusion proteins and comprising an agent of interest with an affinity for immune cells may be used to recruit immune cells, like NK or T-cells, to the target cell that is recognized by the svd-TCR.

In another non-limiting example, T-cells which express svd-TCRs on their cell surface may be used to target cells recognized by the svd-TCR, for example, cancer cells, bacterial cells, virally-invaded cells and other undesired cells, for destruction by the host immune system. Accordingly, svd-TCRs may used in adoptive cell transfer therapy, e.g. similar to chimeric antigen receptors in T-cell based therapies. Because svd-TCRs can comprise a small modular binding domain, they have great flexibility in application as fusion proteins as compared to traditional TCRs which involve two different chains.

Cells may alternatively be redirected to particular organs or sites of healing or sites of inflammation, for example. In a non-limiting example, stem cells may be directed to organs or other microenvironments.

Certain embodiments relate to administering a composition as disclosed herein to a subject which comprises the cell having the epitope of interest.

Certain embodiments relate to a method of identifying a svd-TCR which specifically binds to a peptide bound in a MHC (pMHC), the svd-TCR comprising a first TCR variable domain which specifically binds to the peptide in the absence of a second TCR variable domain. The method comprises: providing a pool of eukaryotic cells which externally present a plurality of unique svd-TCRs that have different variable domains; contacting the pool of eukaryotic cells with two different pMHCs, wherein the two different pMHCs have the same major histocompatibility complex (MHC) but different peptides, one of the different peptides being the desired peptide, and wherein each of the two different pMHCs is labeled with a distinguishable marker; identifying a cell that binds to one of the two different pMHCs and does not bind to the other of the two different pMHCs; and identifying from the identified cell the svd-TCR which specifically binds the desired peptide in the pMHC.

In some embodiments, identifying from the identified cell the svd-TCR which specifically binds the desired peptide in the pMHC comprises isolating from the identified cell the svd-TCR which specifically binds the desired peptide in the pMHC.

The distinguishable markers may be any compound or complex that permits the different peptides to be identified. For example, the distinguishable markers may be fluorescent markers. Many fluorescent markers are known and commercially available, including without limitation Alexa Fluor™ fluorescent dyes (e.g. Alexa Fluor™ 647), phycoerythrin (PE) and the like. For example, but without limitation, biotinylated MHC may be used to attach streptavidin conjugated marker (e.g. a straptavidin conjugated fluorescent marker).

The eukaryotic cells may be vertebrate cells. The eukaryotic cells may be mammalian cells. The eukaryotic cells may be human cells or a human-derived cell line (e.g. HEK293 and the like).

In some embodiments, the plurality of unique TCR variable domains in the method is at least 10 million to 1 billion or more unique TCR variable domains, or any number in between. For example, the plurality of TCR variable domains may be at least 10 million, 12 million, 14 million, 16 million, 18 million, 20 million, 25 million, 30 million, 40 million, 50 million, 60 million, 70 million, 80 million, 90 million, 100 million, 150 million, 200 million, 250 million, 300 million, 350 million, 400 million, 500 million 600 million, 700 million, 800 million, 900 million, 1 billion or 10 billion unique TCR variable domains. The plurality of TCR variable domains may be more than 10 billion unique TCR variable domains.

In some embodiments, the plurality of unique TCR variable domains may differ in the amino acid sequence of CDR1. The plurality of unique TCR variable domains may differ in the amino acid sequence of CDR2. The plurality of unique TCR variable domains may differ in the amino acid sequence of CDR3.

In some embodiments, the svd-TCR may be any svd-TCR or subset of svd-TCRs defined herein, including without limitation single TCR chains and fusion proteins comprising the svd-TCR. For example, the svd-TCR may be a single a TCR chain, a single β TCR chain, a single γ TCR chain, or a single δ TCR chain. The svd-TCR may be a svd-TCR-Fc fusion or any other fusion protein defined herein.

In some embodiments, the method further comprises generating the pool of eukaryotic cells which externally present a plurality of unique svd-TCRs that have different variable domains using in vitro V(D)J recombination. The pool may alternatively be generated using any other known method, including without limitation mutagenesis and/or the use of double-stranded breaks together with Tdt, such as with restriction enzymes, CRISPR, Zinc Finger or Talon methods, or the use of error prone PCR, degenerate oligos or degererate gene synthesis products. Insertions and/or deletions may be produced as a result of in vitro V(D)J recombination methods or from the in-vitro action of TdT and recombination and DNA repair enzymes (e.g. one or more of Artemis nuclease, NDA-dependent protein kinase (DNA-PK), X-ray repair cross-complementing protein 4 (XRCC4), DNA ligase IV, non-homologous end joining factor 1 (NHEJ1), PAXX, and DNA polymerases λ and μ). A double stranded break in DNA may be introduced prior to in vitro use of the above recombination and DNA repair enzymes.

In some embodiments, the pool is generated by producing a plurality of in vitro V(D)J recombination substrates in recombination competent host cells and culturing the host cells in vitro under conditions allowing recombination of recombination signal sequences (RSSs) according to the 12/23 rule. Recombination competent host cells are known in the art and must be capable of expressing RAG-1 and RAG-2 or recombination-functional fragments thereof; non-limiting examples of such host cells are described in WO 2009/129247, WO 2013/134880 and Example 1 of this application. The recombination substrates may comprise an upstream promoter (e.g. CMV promoter and the like), a TCR β or δ variable gene segment followed by a first RSS, a spacer, a second RSS capable of recombining with the first RSS, a TCR diversity gene segment, a third RSS, a spacer, a fourth RSS capable of recombining with the third RSS and a TCR joining gene segment. The recombination substrates may comprise or further comprise an upstream promoter (e.g. CMV promoter and the like), a TCR α or γ variable gene segment followed by a first (or fifth) RSS, a spacer, a second (or sixth) RSS capable of recombining with the first (or fifth) RSS and a TCR joining gene segment.

The TCR joining gene segment may further be joined to a TCR constant region gene segment (or portion thereof, e.g. an extracellular domain of the constant region) and/or a membrane anchor encoding sequence.

Also provided is a svd-TCR, or a fusion protein comprising the svd-TCR, produced, identified or isolated by the method defined herein.

By screening large repertoires of svd-TCRs it is possible to eliminate those svd-TCRs with low peptide-specificity (i.e. peptide-independent affinity). Neither traditional CARs (chimeric antigen receptors) nor natural TCRs require the same level of affinity as soluble molecules because, as cell surface expressed receptors, binding is avidity driven. As a result, TCR-derived therapeutics requires attention to specificity; non-target binding even at low micromolar avidities may cause undesirable side effects. Previous technologies that have isolated TCRs and monoclonal antibodies with pMHC specificities typically do not evaluate binding at such low levels. The use of certain technologies described herein to express these svd-TCRs and fusion proteins thereof on the cell surface provides a means to directly assess low avidity interactions during screening.

The present invention will be further illustrated in the following non-limiting examples.

### EXAMPLES

In these Examples, in vitro V(D)J recombination was used to generate sequence diversity in TCRs to identify pMHC-binding molecules that were peptide specific.

An unintentional result of this effort was the production of TCR molecules with compromised levels of the TCR α chain compared to the TCR β chain. As a result, TCR β chains were present on the cell surface without the paired TCR α chain. When these repertoires were used to find peptide-specific binders, TCRs without alpha chains that had the desired peptide specificity were unexpectedly found. This result was unanticipated as it was assumed that both TCR chains would be required for binding to the pMHC since the natural TCR utilizes both chains and extensive effort has been devoted to trying to generate a stable version of soluble TCRs which comprise both chains.

### Example 1

### Generation of TCR Diversity in TCR β Chain

Patent application WO 2009/129247 (herein incorporated by reference in its entirety) discloses an in vitro system utilizing V(D)J recombination to generate de novo antibodies in vitro. A related system was utilized herein to generate TCRs by changing the coding sequences to correspond to TCR variable (V), TCR diversity (D) and TCR joining (J) sequences. A HEK293 cell line was engineered with a single integrated LoxP site for targeted integration of recombinant plasmid substrates. The HEK293 cell also had integrated RAG-1, RAG-2 and terminal deoxynucleotidyl transferase (TdT), such that V(D)J recombination was inducible with the addition of tetracycline (e.g. as described in WO 2013/134880, herein incorporated by reference in its entirety). The chromosomal location of the LoxP site was selected to support CRE mediated LoxP insertion and to be optimal for V(D)J recombination as well as for high expression of post-recombination products (e.g. as described in WO 2013/134880).

In order to generate the full human TCR repertoire the TCR variable genes were synthesized based on The International Immunogenetics Information Systems database (www.imgt.org). The TCR beta variable gene segments used were the following: TRBV2*01, TRBV3-1*01, TRBV4-1*01, TRBV4-2*01, TRBV4-3*01, TRBV5-1*01, TRBV5-4*01, TRBV5-5*01, TRBV5-6*01, TRBV5-8*01, TRBV6-1*01, TRBV6-2*01, TRBV6-4*01, TRBV6-5*01, TRBV6-6*01, TRBV6-8*01, TRBV6-9*01, TRBV7-2*01, TRBV7-3*01, TRBV7-4*01, TRBV7-6*01, TRBV7-7*01, TRBV7-8*01, TRBV7-9*01, TRBV9*01, TRBV10-1*01, TRBV10-2*01, TRBV10-3*01, TRBV11-1*01, TRBV11-2*01, TRBV11-3*01, TRBV12-3*01, TRBV12-4*01, TRBV12-5*01, TRBV13*01, TRBV14*01, TRBV15*01, TRBV16*01, TRBV18*01, TRBV19*01, TRBV20-1*01, TRBV24-1*01, TRBV25-1*01, TRBV27*01, TRBV28*01, and TRBV29-1*01.

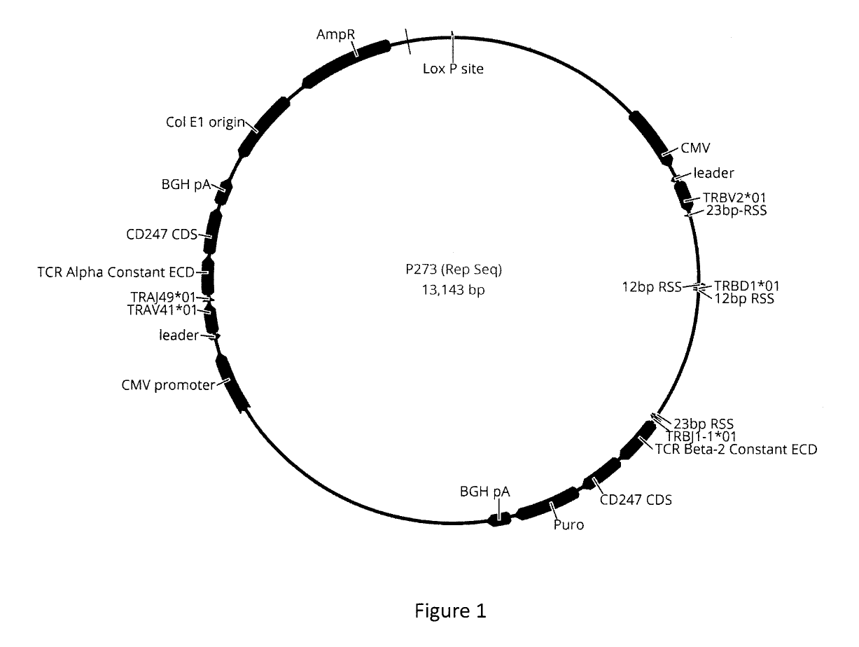

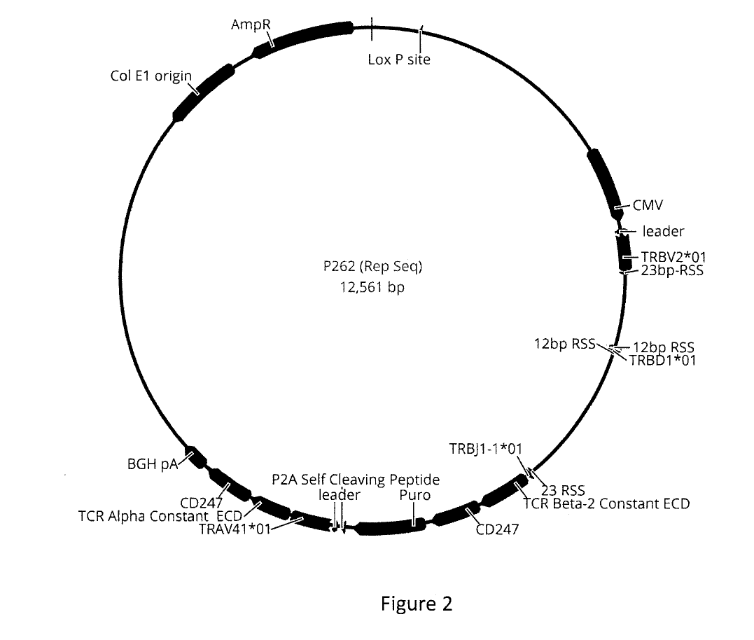

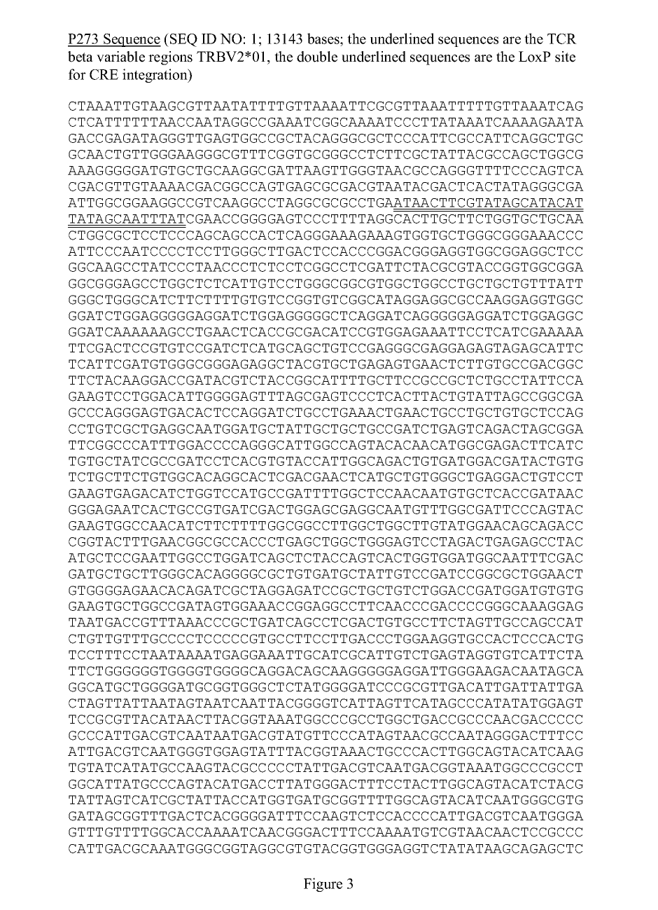

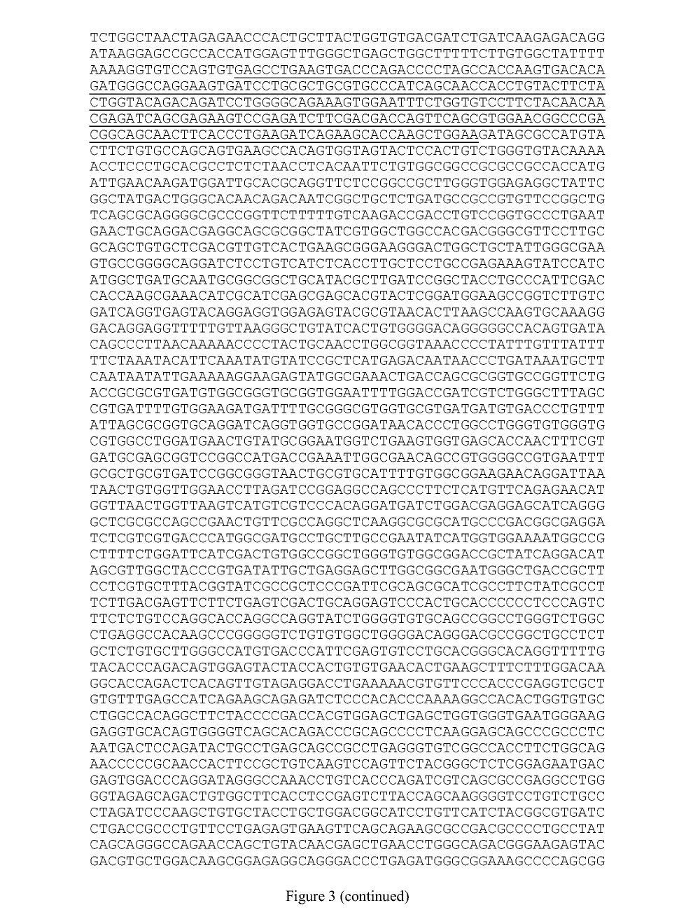

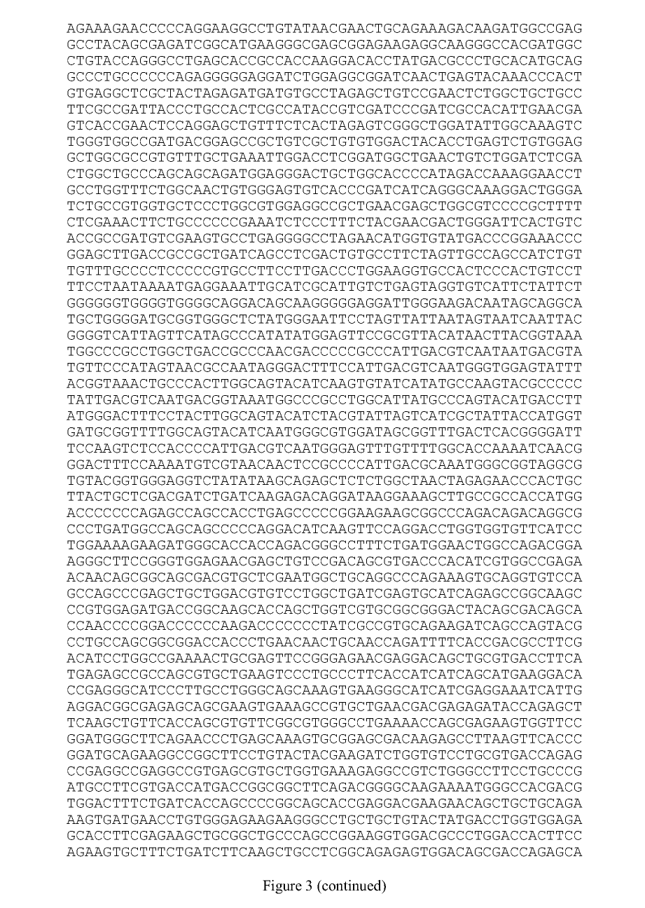

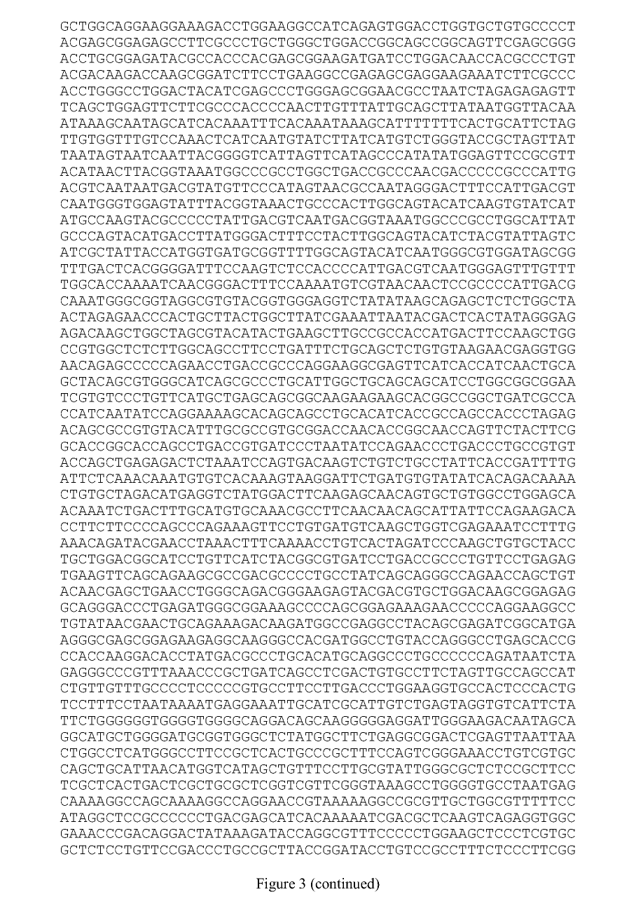

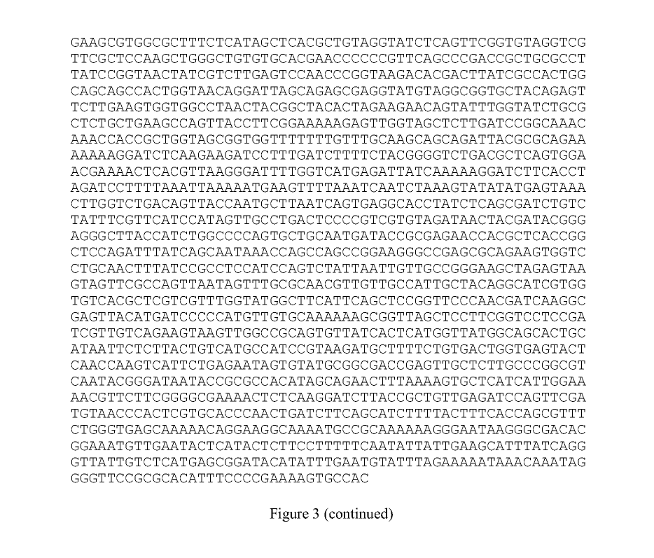

A pool of 46 plasmids each containing a single TCR β variable chain were cloned into a plasmid set containing all of the TCR β joining gene segments. The TCR β diversity segments were subsequently cloned in the previous pool of Vβ and Jβ gene segments. The result was a pool of vector substrates that contained a single TCR β V gene sequence, a single TCR ≢ D gene sequence, and TCR β joining sequences with the appropriate 12bp and 23 bp sequences/spacers. Two exemplary representatives of these sequences are P273 (SEQ ID NO:1) and P262 (SEQ ID NO:2), vector maps for which are respectively shown in FIGS. 1 and 2. The sequence for each of P273 and P262 is shown in FIGS. 3 and 4, respectively. The pool of plasmids were CRE integrated to generate a pool of host cells capable of V(D)J recombination (Fukushige and Sauer 1992 Proc. Natl. Acad. Sci. U.S.A. 89:7905-7909). Cells were subsequently expanded and induced with the addition of tetracycline for V(D)J recombination. Post recombination, each cell expressed a unique TCR β chain expressed in the presence of a single TCR α chain (e.g. P273) or with limited expression of the α chain (e.g. P262).

As shown in FIG. 1, vector P262 (SEQ ID NO:1) has a CMV promoter, a TCR β variable region (shown here as the TRBV2̂01) followed by a 23 base pair recombination signal sequence (RSS), a spacer, a 12 base pair RSS, a TCR D segment (shown here as the TRBD1̂01), a spacer, a 23 base pair RSS, a TCR J gene segment (shown here as TRBJ-1̂01), the TCR beta-2 constant region ECD linked to the CD247 (CD3 zeta) intracellular domain in-frame with puromycin, followed by the bovine growth hormone polyadenylation sequence. On the same plasmid is the TCR α chain. It has its own CMV promoter and is expressed as a independent transcript (TRAV41*01, JRAJ49*01).

The P262 plasmid contains the same tripartite V(D)J structure as P273, however the TCR α chain was not expressed as a separate transcript but instead utilized self-cleaving P2A sequences so that TCR β and TCR α were expressed from the same transcript. The expression of two chains from a single promoter using P2A sequences has been previously described. The constructs disclosed herein, however, had a number of differences which resulted in poor downstream expression of the TCR α chain. These constructs included an additional 718 base pair sequence from the end of the TCR β protein encoding sequence to the end of the P2A coding sequence. The additional length or composition of the puromycin sequence may have had a negative impact on TCR α expression although it is unknown why this occurred. The lack of alpha expression was observed with reduced staining of cells with an anti-TCR alpha specific antibody and was validated by the isolation of recombinant TCR beta chains that did not require the TCR alpha chain for binding.

As shown in FIG. 2, vector P262 (SEQ ID NO: 2) has a CMV promoter, a TCR β variable region (shown here as TRBV2̂01) followed by a 23 base pair recombination signal sequence (RSS), a spacer, a 12 base pair RSS, a TCR D segment (shown here as TRBD1\textcircled{}01), a 12 base pair RSS, a spacer, a 23 base pair RSS, a TCR J gene segment (shown here as TRBJ-1\textcircled{}01), the TCR beta-2 constant region ECD linked to the CD247 (CD3 zeta) intracellular domain in-frame with puromycin, in-frame with P2A sequence, followed by a leader sequence and the same TCR alpha sequences found in P273, followed by the bovine growth hormone polyadenylation sequence.

Vectors P273 and P262 are representative plasmids that were CRE integrated into cells as part of a pool of vectors containing all combinations of the human TCR β V, D and J gene segments. CRE integration resulted in each cell having a single substrate and P262 and P273 show what representative plasmids would be found integrated into the cell line. Following V(D)J recombination each cell generated a unique TCR. All the cells contain the same TCR α chain gene but a different TCR β chain gene. Repertoires of 100 s of millions of TCRs can be effectively generated in this manner.

### Example 2

### Antibody Recognition of pMHC

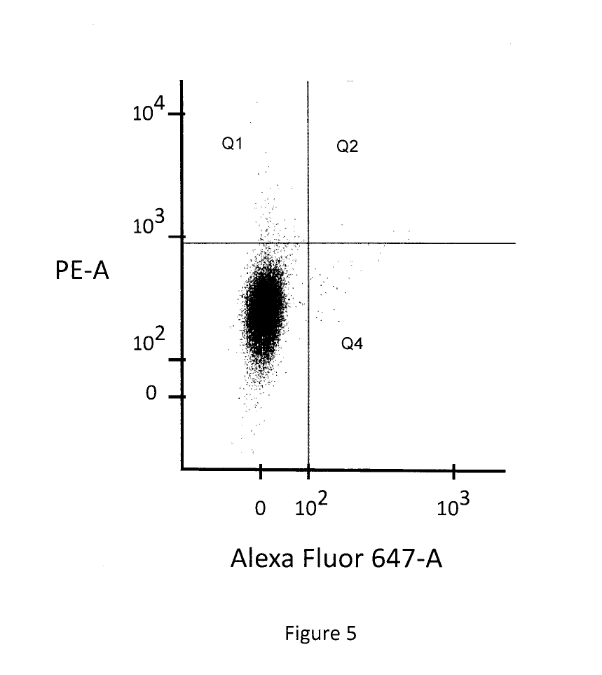

A repertoire of greater than 100 million fully human surface expressed antibodies (prepared as disclosed in WO 2009/129247 and using LoxP integration and tetracycline-induced recombination as per WO 2013/134880) were incubated with a combination of two pMHCs. The MHC:NY-ESO complex was generated with 1 μg/ml Alexa Fluor™ 647-Streptavidin (Jackson, 016-600-084) and 1 μg/mL Biotin-labeled ProS™ MHC Class I A*02:01 SLLMWITQC (NY-ESO; F049-1A-D, available from Prolmmune). The MHC:HIV complex was generated with 1 μg/mL PE-Streptavidin (Jackson, 016-110-084) and Biotin-labeled Pro5™ MHC Class IA*02:01 SLYNTVATL (HIV; F010-1A-D, available from Prolmmune). Following a 1 hour incubation at room temperature, free biotin at 1 μg/mL was added to each reaction and incubation was continued at room temperature for an additional hour. As shown in the FACS plot in FIG. 5, the frequency of antibodies found to be binding to either pMHC is extremely low, less than 0.1% as is observed by the very low number of cells seen in Q1 with high geomeans of PE florescence and as observed by the very low number of cells seen in Q4 with high geomeans of Alexa Fluor™-647 florescence. Antibodies that are able to bind to both complexes would be observed in Q2 as having both high PE florescence and high Alexa Fluor™-647 florescence. Antigen specific binding frequencies from de novo libraries were estimated to be 1 in 500,000 to 1 in a few million and it takes a second or third round of FACS sorting to detect these rare events based on our experience with this in vitro V(D)J system. The inability to see cells at any reasonable frequency therefore is not unexpected. To find antibodies that bind to a specific pMHC is far rarer still given that specificity requires forming contacts with the surface of the exposed peptide in the MHC binding groove as compared to the total exposed surface of the MHC. It should be appreciated that even though anti-MHC antibodies may be recovered they are still infrequent and much more likely to bind to a non peptide-specific region of the MHC.

### Example 3

### TCR Recognition of pMHC

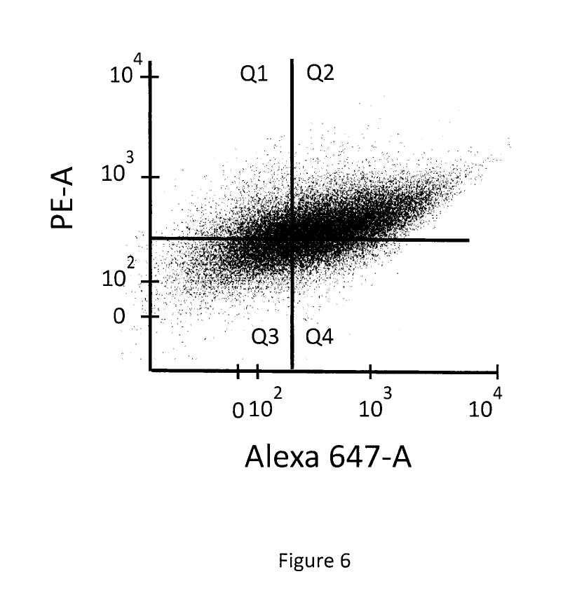

FIG. 6 directly demonstrates, for the first time, that repertoires of non-depleted fully human TCRs are dramatically different than antibodies and have inherent binding affinities for pMHCs.

A FACS plot of binding between a repertoire produced by vector P273 (FIGS. 1 and 3; SEQ ID NO:1) and the same staining conditions using the two different pMHCs as in Example 2. FIG. 6 shows that, unlike antibodies, a large fraction of de novo generated fully human TCRs are binding to two different pMHCs where the difference is only the specific peptides within the complex. In FIG. 6 one no longer sees a traditional population of cells but instead observes a cell population extending into Q2 indicating that a large fraction of cells are able to bind to both complexes, This is in contrast to the antibody data in FIG. 5 (Example 2) where no appreciable binding events were apparent. FIG. 6 shows a total of 1000 events and the vast majority have specificity to both pMHCs. These in vitro de novo TCR repertoires represent different β variable regions each with unique CDR3 sequences paired with the same TCR α chain. This assay is unique in a number of ways. First, it is ex vivo so there was no depletion due to tolerance; in vivo cells that bind to self-peptides are deleted from the repertoire so the repertoire that is observed in the blood will be dramatically impacted by the mechanism of tolerance. Second, unlike phage display approaches, this large repertoire was expressed on the cell surface of a mammalian cell and avidity based interactions which would normally occur on T-cells are approximated. Because the binding interaction is avidity based and the pMHC used here is a soluble pentamer, very low affinities (<1 μM) are easily detectable. The combination of high expression on the cell surface and the multivalent pMHC reagent make the assay extremely sensitive. The combination of utilizing a non-depleted repertoire and characterizing the binding as a cell surface expressed molecule allowed looking at the repertoire in a way that was not previously possible.

### Example 4

### Characterization of a svd-TCR that Specifically Binds a NY-ESO pMHC

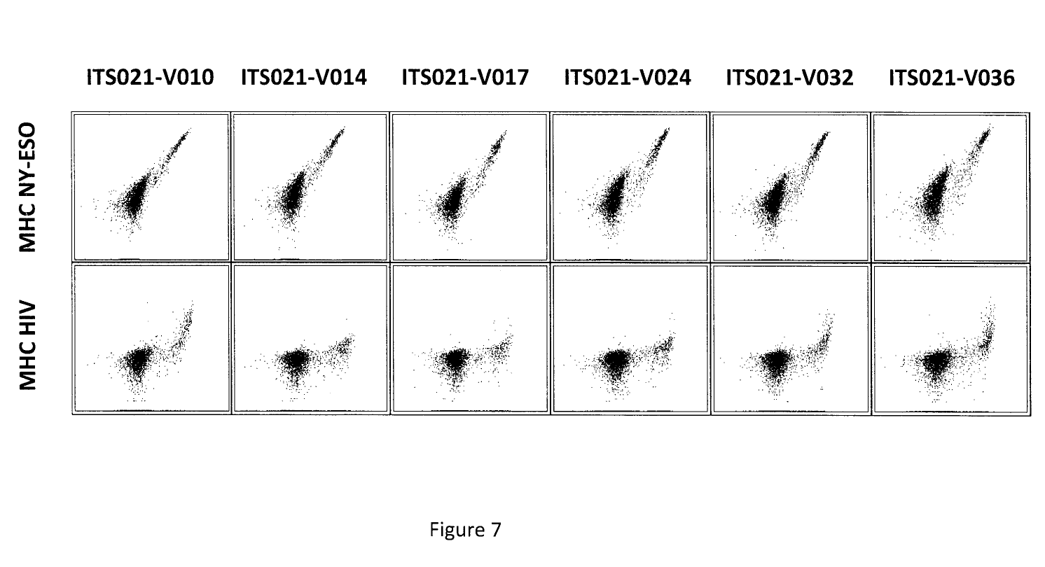

FIG. 7 shows FACS plots used to identify svd-TCRs isolated from a library deficient in TCR a expression which specifically bind to a pMHC that contained the NY-ESO peptide. Staining was performed as described above. The x-axis reflects the amount of the TCR beta chain on the surface of the cell. The y-axis reflects the amount of pMHC complex that is bound to cell. In the upper panel the pMHC complex contains the NY-ESO peptide, in the lower panel the pMHC complex contains an HW peptide. All six clones shown were observed to bind to MHC:NY-ESO. Clones V010, V032 and V036 also showed weaker off target binding to the MHC: HIV as observed in the lower panel. The strength of the binding assay allowed identification of this weak secondary interaction, which is most likely driven by inherent contacts of the svd-TCR β variable region with the MHC through non-peptide contacts.

Staining Procedure for FIG. 7. On the day of staining, transfected cells from each well were trypsinized, resuspended with complete media and each equally divided into two microtubes, which were each treated as follows:
Samples were spun down at speed 5 for 1 minute in a microcentrifuge to remove supernatant.
Each sample was resuspended with 200 μL of 1 μg/mL TCR βF1 (8A3) antibody (Mouse IgG1, Life Technologies, TCR1151) in FACS Buffer (2% FBS in PBS) and incubated for 1 hour at room temperature. This antibody binds to the TCR beta chain.
Samples were spun down to remove supernatant, then:
   One replicate from each transfection condition was resuspended with 100 μL of 1 μg/mL Alexa Fluor™ 647-Streptavidin (Jackson, 016-600-084), 1 μg/mL Biotin-labeled Pro 5 MHC Class I A*02:01 SLLMWITQC (NY-ESO; F049-1A-D, available from ProImmune), and 1 μg/mL R-Phyco-APure Goat αMouse IgG (Jackson, 115-115-164).
   The remaining replicate from each transfection condition was resuspended with 100 μL of 1 μg/mL Alexa Fluor™ 647-Streptavidin (Jackson, 016-600-084), Biotin-labeled Pro 5 MHC Class I A*02:01 SLYNTVATL (HIV; F010-1A-D, available from Prolmmune), and 1 μg/mL R-Phyco-Apure Goat αMouse IgG (Jackson, 115-115-164). The goat anti-Mouse IgG-PE reagent is included to bind to the primary antibody recognizing the human TCR beta chain to allow for the quantitation of TCR beta on the cell surface.
Samples incubated for 30 minutes at room temperature.
Samples were spun down to remove supernatant, then each washed once with 500 μl of PBS.
Samples were spun down to remove wash, then each resuspended to 300 μL with PBS and analyzed via Flow Cytometer (BD Accuri).

### Example 5

It is expected that equivalent experiments utilizing only TCR α chain repertoires, TCR γ chain or TCR δ chain repertoires will show that these other chains will also be capable of being used as a scaffold for pMHC binding. Crystal structures have previously demonstrated that contacts between the TCR and MHC can be observed in either of the TCR chains and the current invention demonstrates that the contacts do not require the presence a heterodimer. The crystal structure of a single TCR chain has not been reported. It was not apparent if the heterodimer was important for maintaining conformational integrity and whether a single chain alone had the conformation appropriate to contact the MHC molecule. Without wishing to be bound by theory, the current disclosure suggests that the TCR beta single chain retains the appropriate conformation to make contact with the pMHC and that a complete heterodimer is not required for the structural integrity of the individual chains and that the α, γ and δ TCR chains will also retain their conformations as single chains.

### Example 6

In this experiment, svd-TCRs were engineered as Fc-fusions which retained their Fc recognition and peptide/MHC binding specificities.

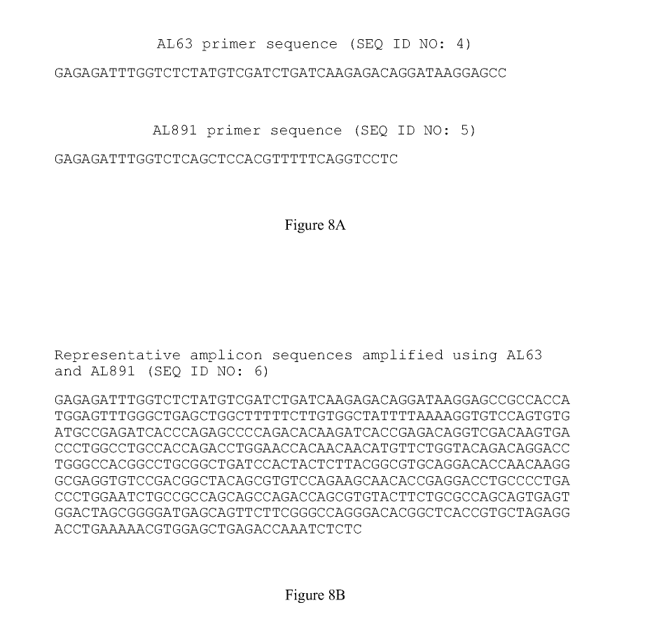

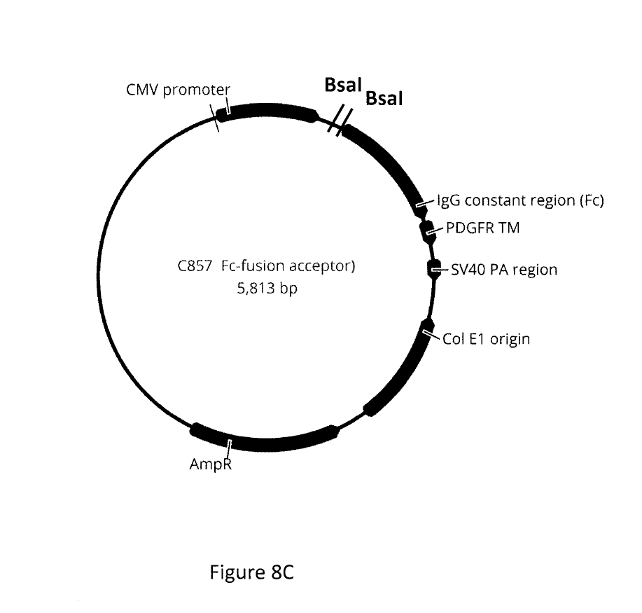

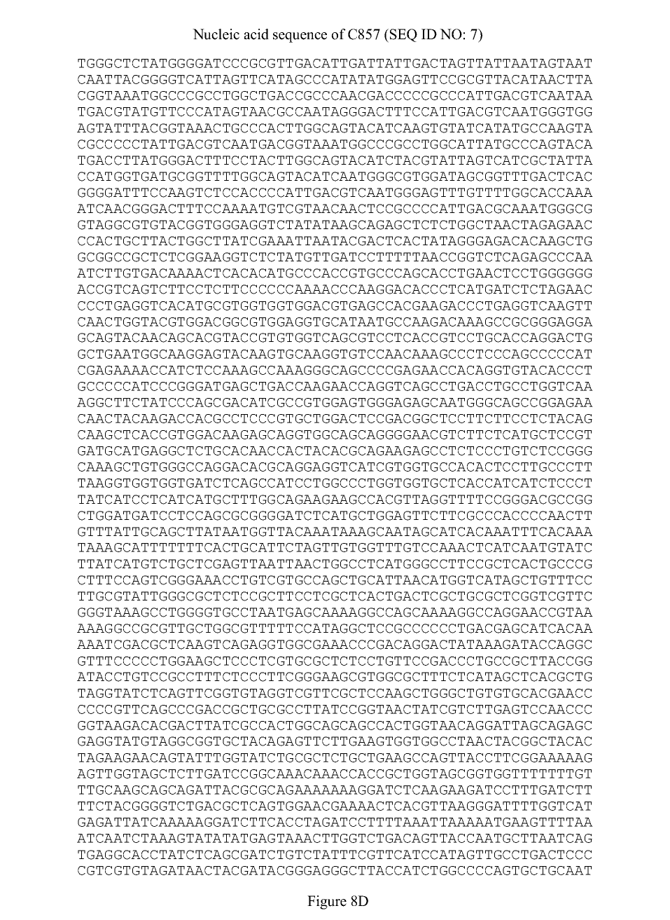

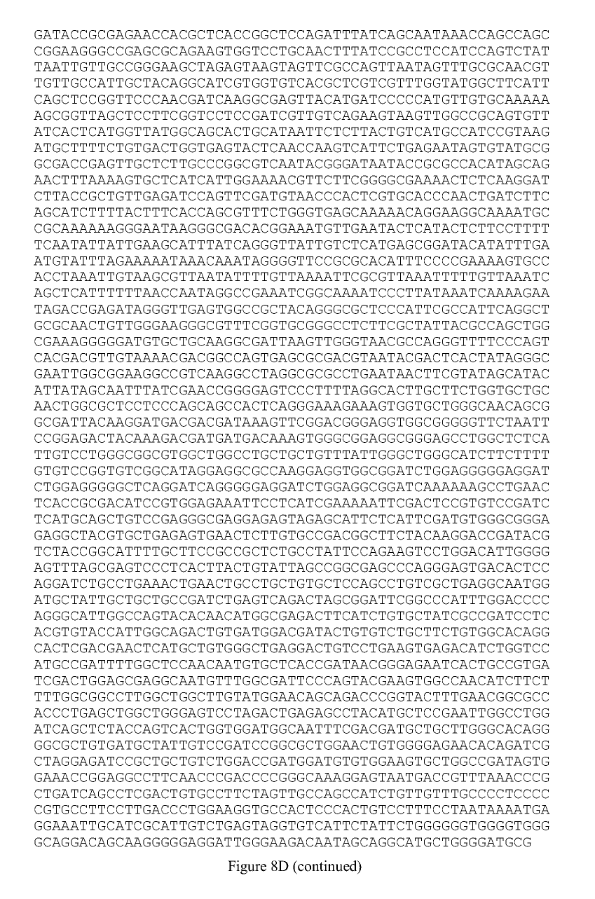

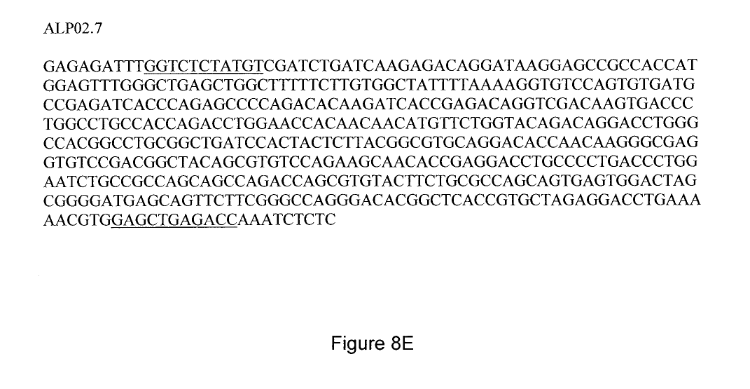

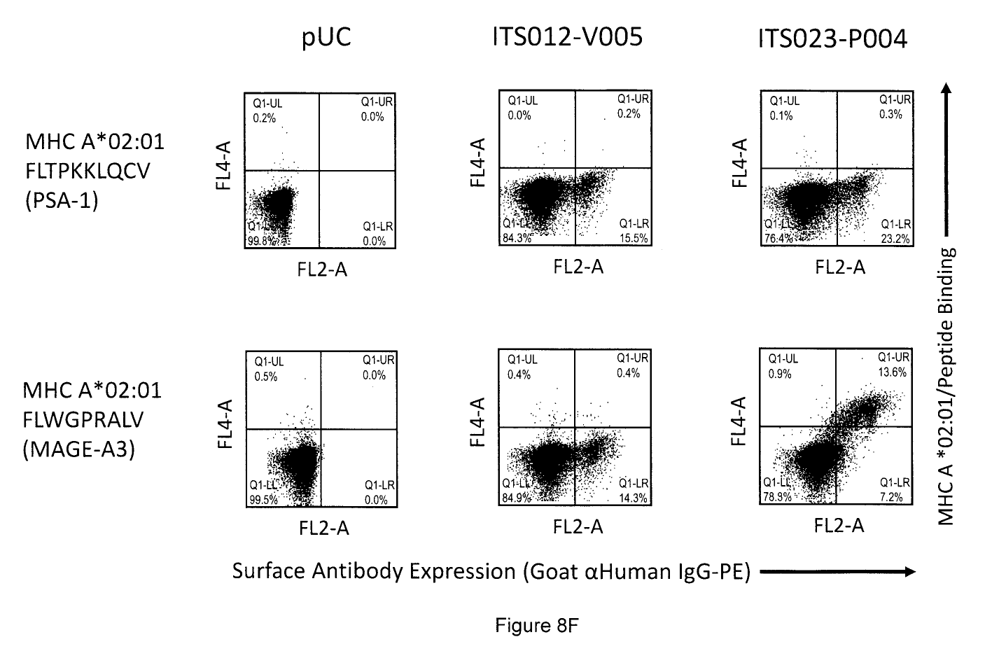

A pool of svd-TCRs was cloned as Fc-fusion proteins. A population of cells that expressed svd-TCRs specific to the MHC/peptide complex containing the MAGE-A3 peptide (amino acid sequence FLWGPRALV; obtained from Proimmune), and not to a negative control peptide bound to the same MHC, were FACS sorted essentially as described in Example #1 and Example #4 for the NY-ESO MHC/peptide complex. FACS sorted, MHC/MAGE-A3 specific cells, 100,000, were lysed and RNA isolated using the QIAgen RNeasy Plus Micro kit for <100,000 cells (QIAGEN 74034) according to the manufacturer's instructions and eluted in the volume specified including 1 μl RNase inhibitor (NEB, M0307S). cDNA synthesis was performed using 2.0 μl of the RNA in a 20 μl final volume reaction contain 0.5 mM dNTPs, 3 μM of RT primer (GAGAGTTTGGATCCCAACTTTCTTGTCCACCTTGGTGTTGC; SEQ ID NO: 3), 10 mM DTT, and 5× Proto Script II buffer and 1.0 μl enzyme (NEB). The svd-TCRs were amplified using KOD DNA polymerase and primers AL63 and AL891 (FIG. 8A; SEQ ID NOs: 4 and 5, respectively). FIG. 8B shows the nucleic acid sequence of a representative PCR product (SEQ ID NO: 6) using AL63 and AL891. AL63 binds to the 5′UTR just upstream of the Kozak sequence common the svd-TCRs shown herein. Reverse primer AL891 anneals to a portion of the TCR beta constant region flanking the J gene segment. The Fc-fusion thus contains the entire TCR beta variable segment, D segment and joining segment as well a few amino acids derived from the TCR beta 2 constant region. The PCR products were then cloned in-frame into the BsaI sites of the surface expressed Fc-acceptor vector C857 (FIGS. 8C and 8D; SEQ ID NO: 7). FIG. 8E shows a schematic of an example amplicon cloned into C857 with BSAI compatible overhangs.

The pool of svd-TCR Fc-fusion proteins were characterized for their ability to be expressed as an Fc fusion protein and the ability to retain their binding specificity. Transfections of HEK293 cells plated in a 24-well plate were carried out with Puc19 (as a negative control), a fully human antibody (ITS012-V005) (as a Fc fusion protein positive control), and the pool of svd-TCR Fc-fusions specific to the MHC/peptide complex containing the MAGE-A3 peptide. Transfections were performed as follows: 400 ng of DNA in 25 μl of PRO293S™ media was combined with 25 μl of PRO293S™ media containing PEI at 1 mg/ml. The complex was vortexed and allowed to form for 20 minutes at room temperature before being applied drop wise to the cells. 24 hours post-transfection the cells were stained. Transfected cells were washed in PBS and then incubated with 100 μl of trypsin and cells were then collected into 500 μl of DMEM. Subsequently the cells are divided into two centrifuge tubes with 250 μl each of the transfected cells. Samples were stained for the display of IgG FC domain using a fluorescent goat anti-human-Fc-PE conjugated polyclonal antibody (Cedarlane, Cat #109-115-098) and for binding to either the MHC/pepitide complex containing MAGE-A3 (FLWGPRALV; obtained from Proimmune) or an irrelevant MHC/peptide containing a peptide derived from PSA-1 (amino acid sequence FLTPKKLQCV; obtained from Proimmune). Biotinylated MHC/peptide complexes were detected by including Alexa Fluor 647 streptavidin (SA647, Invitrogen). The cells were pelleted and then resuspended in either 150 μl of Mage-3 peptide staining solution (containing Goat α-hu-Fc-PE (1:1000)+SA647 (1:2000)+biotinylated MHC:Mage-3A complex (Proimmune Cat #F034-1A-D; 1:100)) or PSA peptide staining solution (containing Goat α-hu-Fc-PE (1:1000)+SA647 (1:2000)+biotinylated MHC:PSA-1 complex (Proimmune Cat #F404-1A-D; 1:100)).

FIG. 8F shows the results of the characterization of the pool of svd-TCRs expressed as Fc fusion proteins.

Transfected cells were simultaneously stained for anti-IgG (Fc) cell surface expression as well as binding to PSA-1 peptide or MAGE-A3 peptide containing MHC/peptide complex. The x-axis reflects the levels of the IgG constant region (Fc) on the cell surface. The y-axis reflects the amount of biotinylated MHC/peptide-SA647 is on the cell surface. The top panels the MHC/peptide complex containing the PSA-1 peptide and the bottom panels are cells stained with the MHC/peptide complex containing MAGE-A3 peptide.

As expected, cells transfected with puc19 did not have any Fc detectable on the cells surface. Puc19 transfected cells also showed no binding to either MHC/peptide complex (see uniform population in the lower left (LL) quadrant of both panels (left) of FIG. 8F). Cells transfected with a membrane expressed fully human antibody (ITS012-V005) showed strong anti-IgG (i.e. anti-Fc) staining (see cells in the lower right quadrant (LR) of both panels (middle) of FIG. 8F). No binding to either MHC/peptide complex is observed with ITS012-V005. The panels (right) show that the pool of svd-TCR Fc-fusions (ITS023-P004) have high levels of anti-IgG expression on the cell surface as indicated by cells staining in the lower right (LR) and upper right (UR) panels of FIG. 8F. The ITS023-P004 cells are specific to the MAGE-A3 MHC/peptide complex as observed as surface positive staining, UR, only in the lower panel and not in the upper panel stained with the PSA-1 MHC/peptide complex. These results demonstrate that the svd-TCRs are expressed at high levels on the cell surface as an Fc-fusion protein and retain their binding specificity as a Fc-fusion protein.

The current disclosure describes, for the first time, a single variable domain T-cell receptor that binds to a specific pMHC and demonstrates that MHC dependent binding does not require a heterodimeric TCR protein complex. This allows targeting cells based on the expression of intracellular proteins which are presented on the cell surface as pMHCs.

Previous to the present disclosure, there was considerable debate in the field as to whether TCRs have evolved to specifically interact with MHC molecules and to recognize pMHCs (Marrack et al. 2008 Annu. Rev. Immunol. 26:171-203). Generating data was complicated by the combination of negative and positive selection that occurs in vivo. This disclosure demonstrates that TCRs from non-selected repertoires do in fact have low affinity for MHC complexes and also demonstrates that single domain TCR β chains have an inherent low affinity for pMHCs, making them a robust scaffold for engineering future biologics.

The svd-TCR molecule in addition to being useful in T-cell engineering also provides the foundation for a modality that allows for bi-specific formats for cell redirected therapies similar to bites and darts as well as for a preferred composition for drug development.

As described herein, a single variable domain of a human TCR is able to recognize pMHCs provides for novel compositions useful as human therapeutics. The unexpected observation that a single variable domain has low affinity interactions with the MHC-microglobulin heterodimer suggests that antigen recognition contacts may be inherently part of the scaffold and that focusing on CDR3 repertoires will allow for the identification of a svd-TCR with pMHC specificity. In addition to providing a robust scaffold for generating a targeting modality with pMHC specificity, svd-TCRs are small in size and as a single domain do not require a linker or other engineering and as such offer advantages over traditional soluble TCR formats for generating fusion proteins as well as chimeric receptors (e.g. CARs).

Whereas conventional antibodies recognize secreted proteins or membrane expressed proteins, this disclosure provides the ability to generate molecules that specifically bind to an epitope of interest, for example, pMHCs. This permits routine targeting of a cell beyond only those proteins on the cell surface but potentially any protein expressed by the cell whether in the nucleus, cytoplasm, mitochondria, golgi, endoplasmic reticulum or any other organelle. This may enable therapeutic interventions not possible with conventional antibodies. In fact, a cell may even be targeted based on the secreted molecules it expresses (antibodies can bind to the secreted molecule but not to the cell that secreted the molecule).

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A single variable domain T-cell receptor (svd-TCR) comprising a first TCR variable domain, the first TCR variable domain specifically binding to an epitope in the absence of a second TCR variable domain, wherein the epitope is not a superantigen.

2. The svd-TCR of claim 1, wherein the first TCR variable domain comprises a TCR Vα domain, a TCR Vβ domain, a TCR Vγ domain or a TCR Vδ domain.

3. The svd-TCR of claim 1, which comprises a TCR α chain, a TCR β chain, a TCR γ chain or a TCR δ chain.

4. The svd-TCR of claim 1, wherein the first TCR variable domain is a human TCR variable domain.

5. The svd-TCR of claim 1, wherein the epitope is a peptide bound in a major histocompatibility complex (MHC) to form a MHC:peptide complex (pMHC).

6. The svd-TCR of claim 5, wherein the MHC is a class I MHC or a class II MHC.

7. The svd-TCR of claim 1, which is fused to an antibody Fc.

8. The svd-TCR of claim 1, which is part of a soluble fusion protein.

9. The svd-TCR of claim 8, wherein the soluble fusion protein comprises: an anticancer agent; a therapeutic radionuclide; a cytotoxic protein; a marker; a purification tag; or a combination thereof.

10. The svd-TCR of claim 1, which is fused to a membrane anchor.

11. The svd-TCR of claim 1, which is part of a chimeric antigen receptor.

12. A composition comprising the svd-TCR of claim 1, or a fusion protein comprising the svd-TCR, and a pharmaceutically acceptable excipient.

13. A cell comprising a nucleic acid sequence encoding the svd-TCR of claim 1 or a fusion protein comprising the svd-TCR, wherein the nucleic acid sequence is in operative association with a promoter and terminator for expression of the svd-TCR.

14. The cell of claim 14, which is a human T-cell or NK cell.

15. A composition comprising the cell of claim 13 and a pharmaceutically acceptable excipient.

16. A method of identifying a single variable domain T-cell receptor (svd-TCR) which specifically binds to a desired peptide bound in a major histocompatibility complex (pMHC), the svd-TCR comprising a first TCR variable domain which specifically binds to the peptide in the absence of a second TCR variable domain, the method comprising:
providing a pool of eukaryotic cells which externally present a plurality of unique svd-TCRs that have different variable domains;
contacting the pool of eukaryotic cells with two different pMHCs, wherein the two different pMHCs have the same major histocompatibility complex (MHC) but different peptides, one of the different peptides being the desired peptide, and wherein each of the two different pMHCs is labeled with a distinguishable marker;
identifying a cell that binds to one of the two different pMHCs and does not bind to the other of the two different pMHCs; and
isolating from the cell the svd-TCR which specifically binds the desired peptide in the pMHC.

17. The method of claim 16, wherein the plurality of unique TCR variable domains comprises at least 10 million unique TCR variable domains.
